SEQUENCE LISTING (24 sequences)

SEQ ID NO: 1 - protein, 107 aa, Mus musculus
QIVLSQSPAILSASPGEKVTMTCRASSSVSYMHWYQQKPGSSPKPWIYAPSNLASGVPARFSGSGSGTSYSLTISRVEAEDAATYYCQQWSFNPPTFGAGTKLELKR

SEQ ID NO: 2 - protein, 107 aa, Artificial sequence
  sequence is synthesized
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKR

SEQ ID NO: 3 - protein, 108 aa, Artificial Sequence
  sequence is synthesized
DIQMTQSPSSLSASVGDRVTITCRASQSISNYLAWYQQKPGKAPKLLIYAASSLESGVPSRFSGSGSGTDFTLTISSLQPEDFATYYCQQYNSLPWTFGQGTKVEIKR

SEQ ID NO: 4 - protein, 10 aa, Mus musculus
RASSSVSYMH

SEQ ID NO: 5 - protein, 7 aa, Mus musculus
APSNLAS

SEQ ID NO: 6 - protein, 9 aa, Mus musculus
QQWSFNPPT

SEQ ID NO: 7 - protein, 122 aa, Mus musculus
QAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVSS

SEQ ID NO: 8 - protein, 122 aa, Artificial sequence [sequence omitted]

SEQ ID NO: 9 - protein, 119 aa, Artificial Sequence
  sequence is synthesized
EVQLVESGGGLVQPGGSLRLSCAASGFTFSSYAMSWVRQAPGKGLEWVAVISGDGGSTYYADSVKGRFTISRDNSKNTLTLQMNSLRAEDTAVYYCARGRVGYSLYDYWGQGTLVTVSS

SEQ ID NO: 10 - protein, 10 aa, Mus musculus
GYTFTSYNMH

SEQ ID NO: 11 - protein, 17 aa, Mus musculus
AIYPGNGDTSYNQKFKG

SEQ ID NO: 12 - protein, 13 aa, Mus musculus
VVYYSNSYWYFDV

SEQ ID NO: 13 - protein, 213 aa, Artificial sequence
  sequence is synthesized
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 452 aa, Artificial sequence
  sequence is synthesized
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 213 aa, Artificial sequence
  sequence is synthesized
DIQMTQSPSSLSASVGDRVTITCRASSSVSYLHWYQQKPGKAPKPLIYAPSNLASGVPSRFSGSGSGTDFTLTISSLQPEDFATYYCQQWAFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 452 aa, Artificial sequence
  sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSYRYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 451 aa, Artificial sequence
  sequence is synthesized
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 107 aa, Artificial sequence
  sequence is synthesized
DIQMTQSPSSLSASVGDRVTITCRASSSVSYLHWYQQKPGKAPKPLIYAPSNLASGVPSRFSGSGSGTDFTLTISSLQPEDFATYYCQQWAFNPPTFGQGTKVEIKR

SEQ ID NO: 19 - protein, 122 aa, Artificial sequence
  sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSYRYWYFDVWGQGTLVTVSS

SEQ ID NO: 20 - protein, 451 aa, Artificial sequence
  sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSYRYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 21 - protein, 10 aa, Artificial sequence
  sequence is synthesized
RASSSVSYXH

SEQ ID NO: 22 - protein, 9 aa, Artificial sequence
  sequence is synthesized
QQWXFNPPT

SEQ ID NO: 23 - protein, 17 aa, Artificial sequence
  sequence is synthesized
AIYPGNGXTSYNQKFKG

SEQ ID NO: 24 - protein, 13 aa, Artificial sequence
  sequence is synthesized
VVYYSXXYWYFDV

(19) United States
(12) Patent Application Publication
(10) Pub. No.: US 2006/0233797 A1
(43) Pub. Date: Oct. 19, 2006

(54) Treatment of inflammatory bowel disease (IBD)

(75) Inventor: Sheila Gujrathi, San Francisco, CA (US)

Correspondence Address: GENENTECH, INC., 1 DNA WAY, SOUTH SAN FRANCISCO, CA, 94080, US

(73) Assignee: GENENTECH, INC.

(21) Appl. No.: 11/403,462

(22) Filed: Apr. 13, 2006

Related U.S. Application Data
(60) Provisional application No. 60/671,902, filed on Apr. 15, 2005.

(51) Int. Cl. A61K 39/395

(52) U.S. Cl. 424/144.1

(57) ABSTRACT

The present invention concerns treatment of IBD, especially ulcerative colitis (UC), with an antibody that binds to CD20.

This is a non-provisional application claiming priority under 35 USC §119 to provisional application No. 60/671,902 filed Apr. 15, 2005, the-entire disclosure of which is hereby incorporated by reference.

### FIELD OF THE INVENTION

The present invention concerns treatment of IBD, especially ulcerative colitis (UC), with an antibody that binds to CD20.

### BACKGROUND OF THE INVENTION

### Inflammatory Bowel Disease (IBD)

Inflammatory bowel disease (IBD) is the name of a group of disorders that cause the intestines to become inflamed. Symptoms of IBD include abdominal cramps and pain, diarrhea, weight loss and intestinal bleeding. The current consensus opinion regarding the pathogenesis of IBD centers on the role of genetically determined dysregulation in the host immune response toward the resident bacterial flora (Pallone et al., The immune system in inflammatory bowel disease. In: Satsangi J, Sutherland L R, editors. Inflammatory Bowel Disease. Spain: Churchill Livingstone, 85-93 (2003)).

Crohn's disease and ulcerative colitis (UC) are the most common forms of IBD.

Crohn's disease usually causes ulcers along the length of the small and large intestines. Crohn's disease generally either spares the rectum, or causes inflammation or infection with drainage around the rectum.

Almost without exception, UC involves the rectum and spreads proximally to contiguous portions or to all of the colon. Disease activity is usually intermittent, with relapses and periods of quiescence. The sigmoidoscopic or colonoscopic picture is characteristic. In mild disease, the colonic mucosa appears hyperemic and granular. In more severe disease, tiny punctuate ulcers are present and the mucosa is characteristically friable and may bleed spontaneously. Histologically, the inflammatory cell infiltrate in active disease usually includes neutrophils, often invading crypts as well as being associated with epithelial damage and crypt distortion. An increased number of lymphocytes in the lamina propria and basal plasmacytosis are usually present.

Between 500,000 and 700,000 patients suffer from UC in the United States (Loftus, Gastroenterology 126:1504-1517(2004)). Extra-colonic manifestations of UC include arthritis, uveitis, aphthous stomatitis, pyoderma gangrenosum, and erythema nodosum. Initial therapy for patients with mild to moderate disease is usually an aminosalicylate. In controlled trials, disease improvement by. various criteria occurred in up to 30% of subjects in the placebo groups; thus, no specific treatment may be an option for patients with very mild disease. Distal left-sided UC involving the rectum and sigmoid colon may be efficaciously treated with 5-aminosalicylate (5-ASA) enema formulations. In patients with active UC who do not respond to standard 5-ASA treatment and in those with more severe disease, oral corticosteroids have been the mainstay of acute symptomatic therapy. However, corticosteroids are not effective in long-term maintenance of remission in patients with UC given that their use is associated with significant toxicity over time (Lennard-Jones et al., Lancet 1:188-189 (1965)).

Patients who do not respond to 5-ASA drugs and corticosteroids for disease exacerbations have limited therapeutic options available. Many of these patients are treated with immunosuppressive agents, most commonly 6-mercaptopurine (6-MP) or azathioprine, which may have a significant delay in onset of therapeutic effect in active disease. In patients with severe disease who have not responded to high-dose IV corticosteroids and who are awaiting colectomy, significant short-term efficacy with IV cyclosporine has been observed in one small placebo-controlled study (Lichtiger et al., N Engl J Med 330:1841-1845 (1994)). Ultimately, colectomy is necessary in 25%-40% of patients. A clear unmet need exists for a safe and effective therapeutic agent that can provide rapid control of active disease and induce prolonged disease remission.

Although the pathogenesis of UC is not fully understood, there is increasing evidence that UC may be an autoimmune disorder, with B cells playing a role in disease pathophysiology. B cells, as well as T cells, are present in basal lymphoid aggregates, a histopathologic feature considered indicative of UC and seen in histologic sections from patients with active UC (Yeung et al., Gut 47:212-227(2000)). In evaluating clinical and histologic parameters that might predict relapse in patients with quiescent UC, the presence of increased numbers of plasma cells in the basal portion of the mucosa was found to be an independent predictor of relapse (Bitton et al., Gastroenterology 120:1320 (2001)). Whereas mucosal inflammation in UC is thought to be driven by activated T cells, these patients have a T-helper-2 (Th2) cytokine expression pattern profile (Monteleone et al., Gut 50(Suppl III)64 (2002)). As. Th2 cytokines classically drive B-cell immune responses and antibody production, a central role for B cell may be postulated in UC.

Increased amounts of IgG, IgM, and IgA and plasma cells, as well as increased production of antibodies against intestinal luminal antigens and autoantigens, have been found in the lamina propria of inflamed colonic mucosa in patients with UC (MacDermott et al., Gastroenterology 81:844-852 (1981)). In addition, data are accumulating on the presence of autoantibodies in patients with UC, although a definite role for these antibodies in the pathogenesis of UC is not certain. Approximately two-thirds of UC patients have a circulating antibody known as perinuclear antineutrophil cytoplasmic antibody (p-ANCA), which is directed against components of neutrophil leukocytes (Quinton et al., Gut 42:788-7-91 (1998)). It has recently been shown that the p-ANCA that occurs in some forms of vasculitis, and which is directed against a different neutrophil component (myeloperoxidase), is itself the cause of vasculitis and tissue damage in experimental animal models of vasculitis (Xiao et al., J Clin Invest 110:955-963 (2002)).

Another marker of autoimmunity is the colonic mucosal B-cell response against human tropomyosin isoform 5 (hTM5), a putative autoantigen in UC. The colonic mucosa of patients with UC had a highly statistically significantly increase in the number of lamina propria B cells that produce IgG against hTM5 compared with patients with Crohn's colitis and non-IBD patients, suggesting an important, distinct role for anti-hTM5 antibodies in UC (Onuma et al., Clin Exp Immunol 121:466-471 (2000)). Similarly, the number of anti-hTM5 IgG immunocytes was significantly higher in patients with UC compared with non-IBD controls, with 21 of 23 patients (91%) having IgG-producing immunocytes, irrespective of clinical activity (Onuma et al., Clin Exp Immunol 121:466-471 (2000)). In addition, the anti-hTM5 antibody has been detected in sera of patients with UC and primary sclerosing cholangitis (Sakimaki et al., Gut 47:236-241 (2000)). It has been demonstrated that anti-colon antibodies in the sera from patients with UC can react with surface antigens in colonic epithelial cells or colonic mucin in goblet cells (Inoue et al., Gastroenterology 121:1523 (2001)). These antibodies may contribute to the destruction of colonic mucosa through antibody-dependent cell-mediated cytotoxic mechanisms against colonic epithelial cells.

In one study, the spontaneous chronic colitis that occurs in mice deficient in T-cell receptor (TCR)α was observed to be more severe in the absence of mature B cells. TCRα-deficient mice with chronic colitis that are crossed with αμ knockout mice have offspring that develop a more severe form of colitis than the TCRα-deficient mice. In this study, the increased severity of colitis was not due to pathogenic flora, but to the complete absence of B cells. In the αμ knockout mice, chronic colitis was markedly attenuated after adoptive transfer of peripheral B cells from the TCRα-deficient mice to 3- to 4-week-old αμ-deficient mice prior to the onset of colitis. This suggests a suppressive role for B cells in the development of colitis in these murine models (Mizoguchi et al., Int Immunol 12:597-605 (2000)).

### CD20 Antibodies and Therapy Therewith

Lymphocytes are one of many types of white blood cells produced in the bone marrow during the process of hematopoiesis. There are two major populations of lymphocytes: B lymphocytes (B cells) and T lymphocytes (T cells). The lymphocytes of particular interest herein are B cells.

B cells mature within the bone marrow and leave the marrow expressing an antigen-binding antibody on their cell surface. When a naive B cell first encounters the antigen for which its membrane-bound antibody is specific, the cell begins to divide rapidly and its progeny differentiate into memory B cells and effector cells called “plasma cells”. Memory B cells have a longer life span and continue to express membrane-bound antibody with the same specificity as the original parent cell. Plasma cells do not produce membrane-bound antibody, but instead produce the antibody in a form that can be secreted. Secreted antibodies are the major effector molecules of humoral immunity.

The CD20 antigen (also called human B-lymphocyte-restricted differentiation antigen, Bp35) is a hydrophobic transmembrane protein with a molecular weight of approximately 35 kD located on pre-B and mature B lymphocytes. Valentine et al., J. Biol. Chem. 264(19):11282-11287 (1989) and Einfeld et al., EMBO J. 7(3):711-717 (1988). The antigen is also expressed on greater than 90% of B-cell non-Hodgkin's lymphomas (NHL) (Anderson et al. Blood 63(6):1424-1433 (1984)), but is not found on hematopoietic stem cells, pro-B cells, normal plasma cells, or other normal tissues (Tedder et al. J. Immunol. 135(2):973-979 (1985)). CD20 regulates an early step(s) in the activation process for cell- cycle initiation and differentiation (Tedder et al, supra), and possibly functions as a calcium- ion channel. Tedder et al., J. Cell. Biochem. 14D: 195 (1990).

Given the expression of CD20 in B-cell lymphomas, this antigen can serve as a candidate for “targeting” of such lymphomas. In essence, such targeting can be generalized as follows: antibodies specific to the CD20 surface antigen of B cells are administered to a patient. These anti-CD20 antibodies specifically bind to the CD20 antigen of (ostensibly) both normal and malignant B cells; the antibody bound to the CD20 surface antigen may lead to the destruction and depletion of neoplastic B cells. Additionally, chemical agents or radioactive labels having the potential to destroy the tumor can be conjugated to the anti-CD20 antibody such that the agent is specifically “delivered” to the neoplastic B cells. Irrespective of the approach, a primary goal is to destroy the tumor; the specific approach can be determined by the particular anti-CD20 antibody that is utilized, and thus, the available approaches to targeting the CD20 antigen can vary considerably.

The rituximab (RITUXAN®) antibody is a genetically engineered chimeric murine/human monoclonal antibody directed against the CD20 antigen. Rituximab is the antibody called “C2B8” in U.S. Pat. No. 5,736,137 issued Apr. 7, 1998 (Anderson et al.). Rituximab is indicated for the treatment of patients with relapsed or refractory low-grade or follicular, CD20-positive, B-cell non-Hodgkin's lymphoma. In vitro, rituximab has been demonstrated to mediate complement-dependent cytotoxicity (CDC) and antibody-dependent cellular cytotoxicity (ADCC) and to induce apoptosis (Reff et al., Blood 83(2):435-445 (1994); Maloney et al., Blood 88:637a (1996); Manches et al., Blood 101:949-954 (2003)). Synergy between rituximab and chemotherapies and toxins has also been observed experimentally. In particular, rituximab sensitizes drug-resistant human B-cell lymphoma cell lines to the cytotoxic effects of doxorubicin, CDDP, VP-16, diphtheria toxin, and ricin (Demidem et al., Cancer Chemotherapy & Radiopharnaceuticals 12(3):177-186 (1997)). In vivo preclinical studies have shown that rituximab depletes B cells from the peripheral blood, lymph nodes, and- bone marrow of cynomolgus monkeys. Reff et al., Blood 83:435-445 (1994).

Rituximab has also been- studied in a variety of non-malignant autoimmune disorders, in which B cells and autoantibodies appear to play a role in disease pathophysiology. Edwards et al., Biochem Soc. Trans. 30:824-828 (2002). Rituximab has been reported to potentially relieve signs and symptoms of, for example, rheumatoid arthritis (RA) (Leandro et al., Ann. Rheum. Dis. 61:883-888 (2002); Edwards et al., Arthritis Rheum., 46 (Suppl. 9): S46 (2002); Stahl et al., Ann. Rheum. Dis., 62 (Suppl. 1): OP004 (2003); Emery et al., Arthritis Rheum. 48(9): S439 (2003)), lupus (Eisenberg, Arthritis. Res. Ther. 5:157-159 (2003); Leandro et al. Arthritis Rheum. 46: 2673-2677 (2002); Gorman et al., Lupus, 13: 312-316 (2004)), immune thrombocytopenic purpura (D'Arena et al., Leuk. Lymphoma 44:561-562 (2003); Stasi et al., Blood, 98: 952-957 (2001); Saleh et al., Semin. Oncol., 27 (Supp 12):99-103 (2000); Zaia et aL, Haematolgica, 87: 189-195 (2002); Ratanatharathorn et al., Ann. Int. Med., 133: 275-279 (2000)), pure red cell aplasia (Auner et al., Br. J. Haematol., 116: 725-728 (2002)); autoimmune anemia (Zaja et al., Haematologica 87:189-195 (2002) (erratum appears in Haematologica 87:336 (2002)), cold agglutinin disease (Layios et al., Leukemia, 15: 187-8 (2001); Berentsen et al., Blood, 103: 2925-2928 (2004); Berentsen et al., Br. J. Haematol., 115: 79-83 (2001); Bauduer, Br. J. Haematol., 112: 1083-1090 (2001); Damiani et al., Br. J. Haematol., 114: 229-234 (2001)), type B syndrome of severe insulin resistance (Coll et al., N. Engl. J. Med., 350: 310-311 (2004), mixed cryoglobulinemia (DeVita et al., Arthritis Rheum. 46 Suppl. 9:S206/S469 (2002)), myasthenia gravis (Zaja et al., Neurology, 55: 1062-63 (2000); Wylam et al., J. Pediatr., 143: 674-677 (2003)), Wegener's granulomatosis (Specks et al., Arthritis & Rheumatism 44: 2836-2840 (2001)), refractory pemphigus vulgaris (Dupuy et al., Arch Dermatol., 140:91-96 (2004)), dermatomyositis (Levine, Arthritis Rheum., 46 (Suppl. 9):S1299 (2002)), Sjogren's syndrome (Somer et al., Arthritis & Rheumatism, 49: 394-398 (2003)), active type-II mixed cryoglobulinemia (Zaja et al., Blood, 101: 3827-3834 (2003)), pemphigus vulgaris (Dupay et al., Arch. Dermatol., 140: 91-95 (2004)), autoimmune neuropathy (Pestronk et al., J. Neurol. Neurosurg. Psychiatry 74:485-489 (2003)),. paraneoplastic opsoclonus-myoclonus syndrome (Pranzatelli et al. Neurology 60(Suppl. 1) P05.128:A395 (2003)),.and relapsing-remitting multiple sclerosis (RRMS). Cross et al. (abstract) “Preliminary results from a phase II trial of rituximab in MS” Eighth Annual Meeting of the Americas Committees for Research and Treatment in Multiple Sclerosis, 20-21 (2003).

A Phase II study (WA16291) has been conducted in patients with rheumatoid arthritis (RA), providing 48-week follow-up data on safety and efficacy of rituximab. Emery et al. Arthritis Rheum 48(9):S439 (2003); Szczepanski et al. Arthritis Rheum 48(9):S121 (2003); Edwards et al., N Engl. J. Med. 350:2572-82 (2004). A total of 161 patients were evenly randomized to four treatment arms: methotrexate, rituximab alone, rituximab plus methotrexate, and rituximab plus cyclophosphamide (CTX). The treatment regimen of rituximab was one gram administered intravenously on days 1 and 15. Infusions of rituximab in most patients with RA were well tolerated by most patients, with 36% of patients experiencing at least one adverse event during their first infusion (compared with 36% of patients receiving placebo). Overall, the majority of adverse events was considered to be mild to moderate in severity and was well balanced across all treatment groups. There were a total of 19 serious adverse events across the four arms over the 48 weeks, which were slightly more frequent in the rituximab/CTX group. The incidence of infections was well balanced across all groups. The mean rate of serious infection in this RA patient population was 4.66 per 100 patient-years, which is lower than the rate of infections requiring hospital admission in RA patients (9.57 per 100 patient-years) reported in a community-based epidemiologic study. Doran et al., Arthritis Rheum. 46:2287-2293 (2002).

The reported safety profile of rituximab in a small number of patients with neurologic disorders, including autoimmune neuropathy (Pestronk et aL, supra), opsoclonus-myoclonus syndrome (Pranzatelli et al., supra), and RRMS (Cross et al., supra), was similar to that reported in oncology or RA. In an ongoing investigator-sponsored trial (IST) of rituximab in combination with interferon-P (IFN-β) or glatiramer acetate in patients with RRMS (Cross et aL, supra), 1 of 10 treated patients was admitted to the hospital for overnight observation after experiencing moderate fever and rigors following the first infusion of rituximab, while the other 9 patients completed the four-infusion regimen without any reported adverse events.

Patent publications concerning CD20 antibodies and CD20 binding molecules include U.S. Pat. Nos. 5,776,456, 5,736,137, 5,843,439, 6,399,061, and 6,682,734, as well as US 2002/0197255, US 2003/0021781, US 2003/0082172, US 2003/0095963, US 2003/0147885 (Anderson et al.); US Patent No. 6,455,043, US 2003/0026804, and WO 2000/09160 (Grillo-Lopez, A.); WO 2000/27428 (Grillo-Lopez and White); WO 2000/27433 and US 2004/0213784 (Grillo-Lopez and Leonard); WO 2000/44788 (Braslawsky et aL); WO 2001/10462 (Rastetter, W.); WOO1/10461 (Rastetter and White); WO 2001/10460 (White and Grillo-Lopez); US 2001/0018041, US 2003/0180292, WO 2001/34194 (Hanna and Hariharan); US 2002/0006404 and WO 2002/04021 (Hanna and Hariharan); US 2002/0012665 and WO 2001/74388 (Hanna, N.); US 2002/0058029 (Hanna, N.); US 2003/0103971 (Hariharan and Hanna); US 2002/0009444 and WO 2001/80884 (Grillo-Lopez, A.); WO 2001/97858 (White, C.); US 2002/0128488 and WO 2002/34790 (Reff, M.); WO 2002/060955 (B raslawsky et al.); WO 2002/096948 (Braslawsky et al.); WO 2002/079255 (Reff and Davies); U.S. Pat. No. 6,171,586 and WO 1998/56418 (Lam et al.); WO 1998/58964 (Raju, S.); WO 1999/22764 (Raju, S.); WO 1999/51642, U.S. Pat. No. 6,194,551, U.S. Pat. No. 6,242,195, U.S. Pat. No. 6,528,624 and U.S. Pat. No. 6,538,124 (Idusogie et al.); WO 2000/42072 (Presta, L.); WO 2000/67796 (Curd et al.); WO 2001/03734 (Grillo-Lopez et al.); US 2002/0004587 and WO 2001/77342 (Miller and Presta); US 2002/0197256 (Grewal, I.); US 2003/0157108 (Presta, L.); WO 04/056312 (Lowman et al.); US 2004/0202658 and WO 2004/091657 (Benyunes, K.); WO 2005/000351 (Chan, A.); US 2005/0032130A1 (Beresini et al.); US 2005/0053602A1 (Brunetta, P.); U.S. Pat. Nos. 6,565,827, 6,090,365, 6,287,537, 6,015,542, 5,843,398, and 5,595,721, (Kaminski et aL); U.S. Pat. Nos. 5,500,362, 5,677,180, 5,721,108, 6,120,767, and 6,652,852 (Robinson et al.); U.S. Pat. No. 6,410,391 (Raubitschek et aL); U.S. Pat. No. 6,224,866 and WOOO/20864 (Barbera-Guillem, E.); WO 2001/13945 (Barbera-Guillem, E.); WO 2000/67795 (Goldenberg); US 2003/0133930 and WO 2000/747i8 (Goldenberg and Hansen); US 2003/0219433 and WO 2003/68821 (Hansen et al.); W02004/058298 (Goldenberg and Hansen); WO 2000/76542 (Golay et al.);WO 2001/72333 (Wolin and Rosenblatt); U.S. Pat. No. 6,368,596 (Ghetie et al.); U.S. Pat. No. 6,306,393 and US 2002/0041847 (Goldenberg, D.); US 2003/0026801 (Weiner and Hartmann); WO 2002/102312 (Engleman, E.); US 2003/0068664 (Albitar et al.); WO 2003/002607 (Leung, S.); WO 2003/049694, US2002/0009427, and US 2003/0185796 (Wolin et al.); WO 2003/061694 (Sing and Siegall); US 2003/0219818 (Bohen et aL); US 2003/0219433 and WO 2003/068821 (Hansen et al.); US 2003/0219818 (Bohen et aL); US2002/0136719 (Shenoy et aL); WO 2004/032828 (Wahl et aL); WO 2002/56910 (Hayden-Ledbetter); US 2003/0219433 Al (Hansen et al); WO 2004/035607 (Teeling et aL); US 2004/0093621 (Shitara et al.); WO 2004/103404 (Watkins et al.); WO 2005/000901 (Tedder et al.); US 2005/0025764 (Watkins et aL); WO2005/016969 and US 2005/0069545 Al (Carr et al.); and WO 2005/014618 (Chang et al.). See also U.S. Pat. No. 5,849,898 and EP 330,191 (Seed et al.); EP332,865A2 (Meyer and Weiss); U.S. Pat. No. 4,861,579 (Meyer et aL); US2001/0056066 (Bugelski etal.); and WO 1995/03770 (Bhat et aL);

Publications concerning therapy with rituximab include: Perotta and Abuel, “Response of chronic relapsing ITP of 10 years duration to rituximab” Abstract # 3360 Blood 10(1)(part 1-2): p. 88B (1998); Perotta et al., “Rituxan in the treatment of chronic idiopathic thrombocytopenic purpura (ITP)”, Blood, 94: 49 (abstract) (1999); Matthews, R., “Medical Heretics” New Scientist (7 Apr. 2001); Leandro et aL, “Lymphocyte depletion in rheumatoid arthritis: early evidence for safety, efficacy and dose response” Arthritis and Rheumatism 44(9): S370 (2001); Leandro et al., “An open study of B lymphocyte depletion in systemic lupus erythematosus”, Arthritis and Rheumatism, 46:2673-2677 (2002), wherein during a ²-week period, each patient received two 500-mg infusions of rituximab, two 750-mg infusions of cyclophosphamide, and high-dose oral corticosteroids, and wherein two of the patients treated relapsed at 7 and 8 months, respectively, and have been retreated, although with. different protocols; Weide et al., “Successful long-term treatment of systemic lupus erythematosus with rituximab maintenance therapy” Lupus, 12: 779-782 (2003), wherein a patient was treated with rituximab (375 mg/m²×4, repeated at weekly intervals) and further rituximab applications were delivered every 5-6 months and then maintenance therapy was received with rituximab 375 mg/m² every three months, and a second patient with refractory SLE was treated successfully with rituximab and is receiving maintenance therapy every three months, with both patients responding well to rituximab therapy; Edwards and Cambridge, “Sustained improvement in rheumatoid arthritis following a protocol designed to deplete B lymphocytes” Rheumatology 40:205-211 (2001); Cambridge et al., “B lymphocyte depletion in patients with rheumatoid arthritis: serial studies of immunological parameters” Arthritis Rheum., 46 (Suppl. 9): S1350 (2002); Edwards et aL, “Efficacy and safety of rituximab, a B-cell targeted chimeric monoclonal antibody: A randomized, placebo controlled trial in patients with rheumatoid arthritis. Arthritis and Rheumatism 46(9): S197 (2002); Pavelka et al., Ann. Rheum. Dis. 63: (S1):289-90 (2004); Emery et al., Arthritis Rheum. 50 (S9):S659 (2004); Levine and Pestronk, “IgM antibody-related polyneuropathies: B-cell depletion chemotherapy using rituximab” Neurology 52: 1701-1704 (1999); DeVita et al., “Efficacy of selective B cell blockade in the treatment of rheumatoid arthritis” Arthritis & Rheum 46:2029-2033 (2002); Hidashida et al. “Treatment of DMARD-refractory rheumatoid arthritis with rituximab.” Presented at the Annual Scientific Meeting of the American College of Rheumatology; Oct. 24-29; New Orleans, La. (2002); Tuscano, J. “Successful treatment of infliximab-refractory rheumatoid arthritis with rituximab” Presented at the Annual Scientific Meeting of the American College of Rheumatology; Oct 24-29; New Orleans, La. (2002); “Pathogenic roles of B cells in human autoimmunity; insights from the clinic” Martin and Chan, Immunity 20:517-527 (2004); Silverman and Weisman, “Rituximab Therapy and Autoimmune Disorders, Prospects for Anti-B Cell Therapy” Arthritis and Rheumatism 48: 1484-1492 (2003); Kazkaz and Isenberg, “Anti B cell therapy (rituximab) in the treatment of autoimmune diseases” Current opinion in pharmacology 4: 398-402 (2004); Virgolini and Vanda, “Rituximab in autoimmune diseases” Biomedicine & pharmacotherapy 58: 299-309(2004); Klemmer et al., “Treatment of antibody mediated autoimmune disorders with an antiCD20 monoclonal antibody Rituximab” Arthritis And Rheumatism 48(9):S624-S624 (2003); Kneitz et al., “Effective B cell depletion with rituximab in the treatment of autoimmune diseases” Immunobiology 206: 519-527 (2002); Arzoo et al., “Treatment of refractory antibody mediated autoimmune disorders with an anti-CD20 monoclonal antibody (rituximab)” Annals of the Rheumatic Diseases 61(10):922-4 (2002) Looney, R., “Treating human autoimmune disease by depleting B cells” Ann Rheum Dis. 61: 863-866 (2002); Lake and Dionne, “Future Strategies in Immunotherapy” in Burger's Medicinal Chemistry and Drug Discovery (2003 by John Wiley & Sons, Inc.) Article Online Posting Date: January 15, 2003 (Chapter 2 “Antibody-Directed Immunotherapy”); Liang and Tedder, Wiley Encyclopedia of Molecular Medicine, Section: CD20 as an Immunotherapy Target, article online posting date: 15 Jan. 2002 entitled “CD20”; Appendix 4A entitled “Monoclonal Antibodies to Human Cell Surface Antigens” by Stockinger et al., eds: Coligan et al., in Current Protocols in Immunology (2003 John Wiley & Sons; Inc) Online Posting Date: May, 2003; Print Publication Date: February, 2003; Penichet and Morrison, “CD Antibodies/molecules: Definition; Antibody Engineering” in Wiley Encyclopedia of Molecular Medicine Section: Chimeric, Humanized and Human Antibodies; posted online 15 Jan. 2002; Specks et al. “Response of Wegener's granulomatosis to anti-CD20 chimeric monoclonal antibody therapy” Arthritis & Rheumatism 44:2836-2840 (2001); online abstract submission and invitation Koegh et al., “Rituximab for Remission Induction in Severe ANCA-Associated Vasculitis: Report of a Prospective Open-Label Pilot Trial in 10 Patients”, American College of Rheumatology, Session Number: 28-100, Session Title: Vasculitis, Session Type: ACR Concurrent Session, Primary Category: 28 Vasculitis, Session Oct. 18, 2004 (http:www.abstractsonline.com/viewer/SearchResults.asp); Eriksson, “Short-term outcome and safety in 5 patients with ANCA-positive vasculitis treated with rituximab”, Kidney and Blood Pressure Research, 26: 294 (2003); Jayne et al., “B-cell depletion with rituximab for refractory vasculitis” Kidney and Blood Pressure Research, 26: 294 (2003); Jayne, poster 88 11^{th} International Vasculitis and ANCA workshop), 2003 American Society of Nephrology; Stone and Specks, “Rituximab Therapy for the Induction of Remission and Tolerance in ANCA-associated Vasculitis”, in the Clinical Trial Research Summary of the 2002-2003 Immune Tolerance Network, http:www.immunetolerance.org/research/autoimmune/trials/stone.html; and Leandro et al., “B cell repopulation occurs mainly from naïve B cells in patient with rheumatoid arthritis and systemic lupus erythematosus” Arthritis Rheum., 48 (Suppl 9): S1160 (2003).

### SUMMARY OF THE INVENTION

In a first aspect, the invention concerns a method for treating moderate-severe inflammatory bowel disease (IBD) in a human subject comprising administering to the subject an effective amount of a CD20 antibody, wherein administration of the antibody results in a clinical response or disease remission in the subject.

In another aspect, the invention concerns a method for treating inflammatory bowel disease (IBD) in a human subject with active IBD comprising administering only one or two doses of a CD20 antibody to the subject, wherein disease remission or clinical response is achieved upon administration of the one or two doses of the CD20 antibody.

The invention further provides a method for treating inflammatory bowel disease (IBD) in a human subject with active IBD comprising administering to the subject an effective amount of a CD20 antibody and further comprising administering to the subject an effective amount of a second medicament selected from the group consisting of an aminosalicylate, an oral corticosteroid, 6-mercaptopurine (6-MP) and azathioprine.

In yet a further aspect, the invention relates to a method for reducing a disease activity index (DAI) score in a human subject with active ulcerative colitis (UC) comprising administering a CD20 antibody to the subject in an amount effective to reduce DAI score.

In yet a further aspect, the invention relates to an article of manufacture comprising:
i. a container comprising a CD20 antibody; and
ii. a package insert with instructions for treating inflammatory bowel disease (IBD) in a human subject, wherein the instructions indicate that an effective amount of the CD20 antibody is administered to the human subject.

### BRIEF DESCRIPTION OF THE DRAWINGS

FIG. 1A is a sequence alignment comparing the amino acid sequences of the variable light domain (V_{L}) of each of murine 2H7 (SEQ ID NO:1), humanized 2H7.v16 variant (SEQ ID NO:2), and the human kappa light chain subgroup I (SEQ ID NO:3). The CDRs of V_{L} of 2H7 and hu2H7.v16 are as follows: CDR1 (SEQ ID NO:4), CDR2 (SEQ ID NO:5 ), and CDR3 (SEQ ID NO:6).

FIG. 1B is a sequence alignment comparing the amino acid sequences of the variable heavy domain (V_{H}) of each of murine 2H7 (SEQ ID NO:7), humanized 2H7.v16 variant (SEQ ID NO:8), and the human consensus sequence of the heavy chain subgroup 1 ml (SEQ ID NO:9). The CDRs of V_{H} of 2H7 and hu2H7.v16 are as follows: CDR1 (SEQ ID NO:10), CDR2 (SEQ ID NO:11), and CDR3 (SEQ ID NO:12).

In FIG. 1A and FIG. 1B, the CDR1, CDR2 and CDR3 in each chain are enclosed within brackets, flanked by the framework regions, FR1-FR4, as indicated. 2H7 refers to murine 2H7 antibody. The asterisks in between two rows of sequences indicate the positions that are different between the two sequences. Residue numbering is according to Kabat et al. Sequences of Immunological Interest, 5th Ed. Public Health Service, National Institutes of Health, Bethesda, Md. (1991), with insertions shown as a, b, c, d, and e.

FIG. 2 shows an alignment of the mature 2H7.v16 and 2H7.v5 11 light chains (SEQ ID Nos. 13 and 15, respectively), with Kabat variable domain residue numbering and Eu constant domain residue numbering.

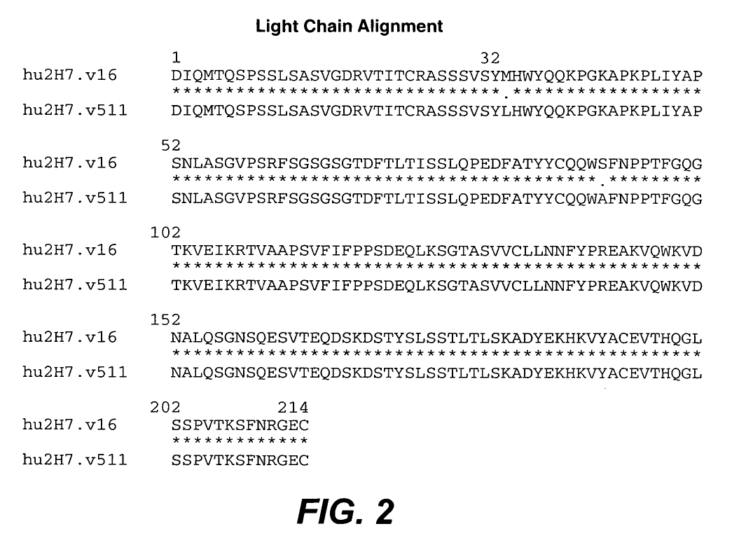

FIG. 3 shows an alignment of the mature 2H7.v16 and 2H7.v511 heavy chains (SEQ ID Nos. 14 and 16, respectively), with Kabat variable domain residue numbering and Eu constant domain residue numbering.

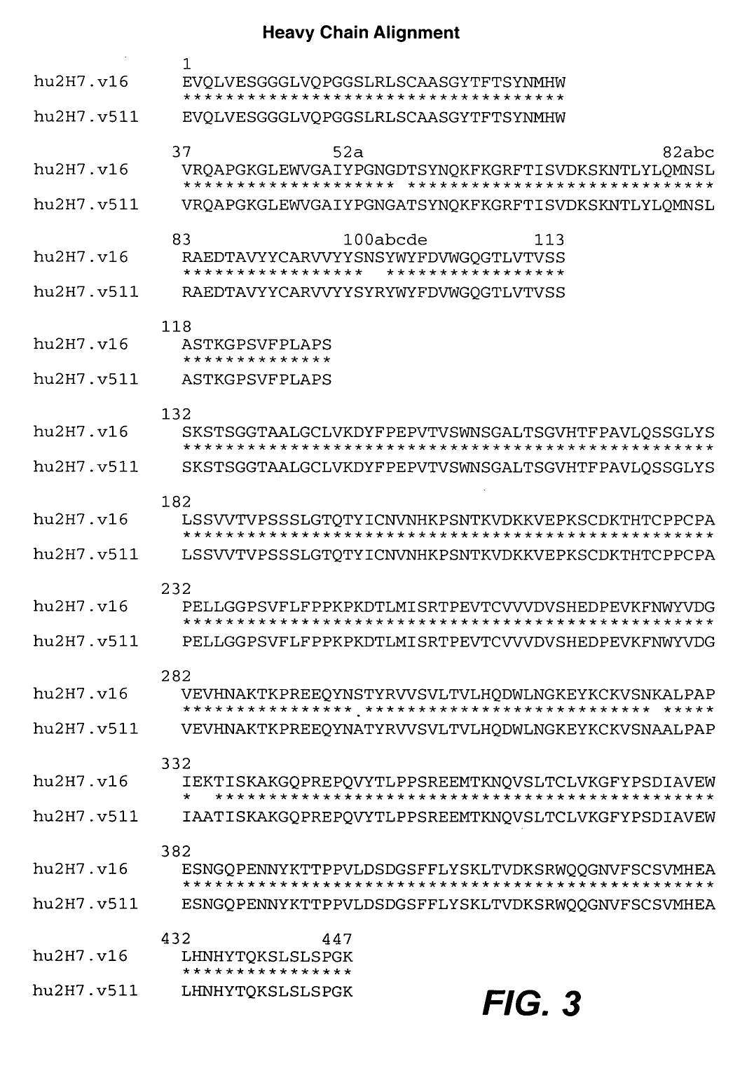

FIG. 4 depicts study schema for the protocol in Example 1.

### DETAILED DESCRIPTION OF THE PREFERRED EMBODIMENTS

I. Definitions

“Inflammatory bowel disease” or “IBD” refers to the group of disorders that cause the intestines to become inflamed, generally manifested with symptoms including abdominal cramps and pain, diarrhea, weight loss and intestinal bleeding. The main forms of IBD are ulcerative colitis (UC) and Crohn's disease.

“Ulcerative colitis” or “UC” is a chronic, episodic, inflammatory disease of the large intestine and rectum characterized by bloody diarrhea. Ulcerative colitis is characterized by chronic inflammation in the colonic mucosa and can be categorized according to location: “proctitis” involves only the rectum, “proctosigmoiditis” affects the rectum and sigmoid colon, “left-sided colitis” encompasses the entire left side of the large intestine, “pancolitis” inflames the entire colon.

“Crohn's disease,” also called “regional enteritis,” is a chronic autoimmune disease that can affect any part of the gastrointestinal tract but most commonly occurs in the ileum (the area where the small and large intestine meet). Crohn's disease, in contrast to ulcerative colitis, is characterized by chronic inflammation extending through all layers of the intestinal wall and involving the mesentery as well as regional lymph nodes. Whether or not the small bowel or colon is involved, the basic pathologic process is the same.

Ulcerative Colitis and Crohn's disease can be distinguished from each other clinically, endoscopically, pathologically, and serologically in more than 90% of cases; the remainder are considered to be indeterminate IBD (Harrison's Principles of Internal medicine, 12^{th} edition, p. 1271 (1991)).

“Moderate-severe” IBD is IBD where the signs or symptoms of disease in the subject are greater than mild. Such subjects can be identified- by a skilled gastroenterologist. The subject with moderate-severe IBD may have been treated with oral corticosteroids for UC within 2 years prior to screening, and/or treatment intensity may have been equal to or greater than a prednisone equivalent dose of 20 mg/day for at least 2 weeks' duration. Such subjects may be steroid refractory and/or steroid-dependent. A subject with moderate-severe UC may be selected based on DAI score, for example, where a DAI score ≧6, ≧2 rectal bleeding score, and/or ≧2 flexible sigmoidoscopy score indicates the subject has moderate-severe UC. Alternatively, or additionally, the criteria for assessment of mild, moderate, and severe disease as in Truelove and Witts Br Med J. 2:1041-1048 (1955) (see Table 1 below) may be used to identify such subjects. Subjects with fulminant or toxic colitis usually have more than 10 bowel movements per day, continuous bleeding, abdominal distention and tenderness, and radiologic evidence of edema and possibly bowel dilation.

TABLE 1
Criteria of Trulove and Witts for Assessing
Disease Activity in Ulcerative Colitis

| | | |
|---|---|---|
| Daily bowel | < or = to 5 | >5 |
| movements (no.) | | |
| Hematochezia | Small amounts | Large amounts |
| Temperature | <37.5° C. | > or = to 37.5° C. |
| Pulse | <90/min | > or = 90/min |
| Erythrocyte | <30 mm/h | > or = to 30 mm/h |
| sedimentation rate | | |
| Hemoglobin | >10 g/dl | < or = to 10 g/dl |

Subjects with fewer than all 6 of the above criteria for severe activity have moderately active disease.

A “subject” herein is a human subject.

A subject with “active” IBD is experiencing at least one symptom of IBD at the time of screening or initial treatment.

“Steroid-refractory” IBD is IBD which progresses, or worsens, even though steroid is being administered to the subject with IBD.

A subject with “steroid-dependent” IBD is dependent on steroid use, and can not taper or withdraw steroid administration due to persistent symptoms.

A “symptom” of IBD is a morbid phenomenon or departure from the normal in structure, function, or sensation, experienced by the subject and indicative of IBD.

“Mucosa” is moist tissue that lines particular organs and body cavities throughout the body, including the gastrointestinal tract. Glands along the mucosa secrete mucus (a thick fluid).

“Colon” is the division of the large intestine extending from the cecum to the rectum.

“Colonic” mucosa is mucosa that lines the colon.

“Peyer's patches” are aggregated lymphatic follicles found throughout the body, especially in the mucous linings of the digestive and respiratory tracts.

By “disease remission” is intended substantially no evidence of the symptoms of disease. Remission may be achieved within a specified time frame, such as within or at about 8 weeks, from the start of treatment with, or from the initial dose of, the antagonist or antibody. Remission may also be sustained for a period of time, such as for ≧24 weeks, or ≧48 weeks. Disease remission may be defined as defined as a sigmoidoscopy score of 0 or 1 and/or rectal bleeding score of 0.

A “sigmoidoscopy” is an inspection, through an endoscope, of the interior of the sigmoid colon.

A “sigmoidoscopy score” refers to a score assigned by a clinician based on a sigmoidoscopy. The preferred sigmoidscopy scoring system is as follows:
0=normal or inactive disease
1=mild disease (erythema, decreased vascular pattern, mild friability)
2=moderate disease (marked erythema, absent vascular pattern, friability, erosions)
3=severe disease (spontaneous bleeding, ulceration)

“Rectal bleeding” refers to any bleeding in or from the rectum.

A “rectal bleeding score” is the score or grade assigned for the extent, if any, of rectal bleeding. A daily bleeding score represents the most severe bleeding of the day. The preferred rectal bleeding scoring system is:
0=no blood seen
1=streaks of blood with stool less than half the time
2=obvious blood with stool most of the time
3=blood alone passed.

By “clinical response” is meant an improvement in the symptoms of disease. The clinical response may be achieved within a certain time frame, for example, within or at about 8 weeks from the start of treatment with, or from the initial dose of, the antagonist or antibody. Clinical response may also be sustained for a period of time; such as for ≧24 weeks, or ≧48 weeks. Clinical response may be evaluated in terms of a reduction in disease activity index (DAI) score, for example, the DAI score may be reduced by greater than or equal to 3 points.

A “disease activity index (DAI)” scoring system is a method for quantitatively assessing UC activity. The preferred DAI scoring system is shown in Table 2 below.

TABLE 2
DAI Scoring System for Assessment of UC Activity

Stool frequency (each subject serves as his/her own control to establish
the degree of abnormality of the stool frequency)
0 = normal number of stools for this subject
1 = 1-2 stools more than normal
2 = 3-4 stools more than normal
3 = 5 or more stools more than normal
Rectal bleeding (the daily bleeding score represented
the most severe bleeding of the day)
0 = no blood seen
1 = streaks of blood with stool less than half the time
2 = obvious blood with stool most of the time
3 = blood alone passed.
Findings of flexible protosigmoidoscopy
0 = normal or inactive disease
1 = mild disease (erythema, decreased vascular pattern)
2 = moderate disease (marked erythema, absent vascular pattern,
friability, erosions)
3 = severe disease (spontaneous bleeding, ulceration)
Physician's global assessment (acknowledges the 3 other criteria, the
subject's daily record of abdominal discomfort and general sense of
well-being, and other observations, such as physical findings and the
subject's performance status)
0 = normal
1 = mild disease
2 = moderate disease
3 = severe disease

An “autoantibody” is an antibody raised by a subject and directed against a subject's own antigen.

A “tropomyosin” is fibrous protein extractable from muscle. There are 8 known human tropomyosin isoforms. In colon epithelial cells, human tropomyosin isoform 5 (hTM5) is the predominant isoform, with lesser amounts of isoform 4 (hTM4).

By “anti-hTM5 antibody” is intended autoantibody raised by a subject and directed against that subject's hTM5.

“Perinuclear antineutrophil cytoplasmic antibody (p-ANCA)” refers to autoantibody raised by a subject and directed against components of that subject's neutrophil leukocytes. “Perinuclear” refers to the staining pattern of such autoantibodies.

By “atypical” autoantibody level, is meant a level of such autoantibody that exceeds the normal level. Such normal or typical autoantibody level may be the level found in colonic tissue or mucosa of a normal subject, or subject who is not suffering from IBD.

A “B cell” is a lymphocyte that matures within the bone marrow, and includes a naïve B cell, memory B cell, or effector B cell (plasma cells). The B cell herein may be a normal or non-malignant B cell.

A “B-cell surface marker” or “B-cell surface antigen” herein is an antigen expressed on the surface of a B cell that can be targeted with an antagonist or antibody that binds thereto. Exemplary B-cell surface markers include the CD10, CD19, CD20, CD21, CD22, CD23, CD24, CD37, CD40, CD53, CD72, CD73, CD74, CDw75, CDw76, CD77, CDw78, CD79a, CD79b, CD80, CD81, CD82, CD83, CDw84, CD85 and CD86 leukocyte surface markers (for descriptions, see The Leukocyte Antigen Facts Book, 2^{nd} Edition. 1997, ed. Barclay et aL Academic Press, Harcourt Brace & Co., New York). Other B-cell surface markers include RP105, FcRH2, B-cell CR2, CCR6, P2X5, HLA-DOB, CXCR5, FCER2, BR3, Btig, NAG14, SLGC16270, FcRH1, IRTA2, ATWD578, FcRH3, IRTA1, FcRH6, BCMA, and 239287. The B-cell surface marker of particular interest is preferentially expressed on B cells compared to other non-B-cell tissues of a subject and may be expressed on both precursor B cells and mature B cells.

The “CD20” antigen, or “CD20,” is an about 35-kDa, non-glycosylated phosphoprotein found on the surface of greater than 90% of B cells from peripheral blood or lymphoid organs. CD20 is present on both normal B cells as well as malignant B cells, but is not expressed on stem cells. Other names for CD20 in the literature include “B-lymphocyte- restricted antigen” and “Bp35”. The CD20 antigen is described in Clark et al., Proc. Natl. Acad. Sci. (USA) 82:1766 (1985), for example.

A “B-cell surface marker antagonist” is a molecule that, upon binding to a B-cell surface marker on B cells, destroys or depletes B cells in a subject and/or interferes with one or more B cell functions, e.g. by reducing or preventing a humoral response elicited by the B cell. The antagonist preferably is able to deplete B cells (i.e. reduce circulating B cell levels) in a subject treated therewith. Such depletion may be achieved via various mechanisms such antibody-dependent cell-mediated cytotoxicity (ADCC) and/or complement dependent cytotoxicity (CDC), inhibition of B cell proliferation and/or induction of B cell death (e.g. via apoptosis). Antagonists included within the scope of the present invention include antibodies, synthetic or native-sequence peptides, immunoadhesins, and small-molecule antagonists that bind to a B-cell surface marker such as CD20, optionally conjugated with or fused to a cytotoxic agent. The preferred antagonist comprises an antibody.

A “CD20 antibody antagonist” herein is an antibody that, upon binding to CD20 on B cells, destroys or depletes B cells in a subject and/or interferes with one or more B-cell functions, e.g., by reducing or preventing a humoral response elicited by the B cell. The antibody antagonist preferably is able to deplete B cells (i.e., reduce circulating B-cell levels) in a subject treated therewith. Such depletion may be achieved via various mechanisms such antibody-dependent cell-mediated cytotoxicity (ADCC) and/or complement-dependent cytotoxicity (CDC), inhibition of B-cell proliferation and/or induction of B-cell death (e.g., via apoptosis).

The term “antibody” herein is used in the broadest sense and specifically covers monoclonal antibodies, polyclonal antibodies, multispecific antibodies (e.g. bispecific antibodies) formed from at least two intact antibodies, and antibody fragments so long as they exhibit the desired biological activity. “Antibody fragments” comprise a portion of an intact antibody, preferably comprising the antigen binding region thereof. Examples of antibody fragments include Fab, Fab′, F(ab′)₂, and Fv fragments; diabodies; linear antibodies; single-chain antibody molecules; and multispecific antibodies formed from antibody fragments.

An “intact antibody” herein is one which comprises two antigen binding regions, and an Fc region. Preferably, the intact antibody has a functional Fc region.

Examples of CD20 antibodies include: “C2B8,” which is now called “rituximab” (“RITUXAN®”) (U.S. Pat. No. 5,736,137); the yttrium-[90]-labelled 2B8 murine antibody designated “Y2B8” or “Ibritumomab Tiuxetan” (ZEVALIN®) commercially available from IDEC Pharmaceuticals, Inc. (U.S. Pat. No. 5,736,137; 2B8 deposited with ATCC under accession no. HB 11388 on Jun. 22, 1993); murine IgG2a “B 1,” also called “Tositumomab,” optionally labelled with 1311 to generate the “131I-BI” or “iodine I131 tositumomab” antibody (BEXXAR™) commercially available from Corixa (see, also, U.S. Pat. No. 5,595,721); murine monoclonal antibody “1F5” (Press et al. Blood 69(2):584-591 (1987) and variants thereof including “framework patched” or humanized IF5 (WO 2003/002607, Leung, S.; ATCC deposit HB-96450); murine 2H7 and chimeric 2H7 antibody (U.S. Pat. No. 5,677,180); humanized 2H7 (WO 2004/056312, Lowman et al., and as set forth below); 2F2 (HuMax-CD20), a fully human, high-affinity antibody targeted at the CD20 molecule in the cell membrane of B-cells (Genmab, Denmark; see, for example, Glennie and van de Winkel, Drug Discovery Today 8: 503-510 (2003) and Cragg et al., Blood 101: 1045-1052 (2003); WO 2004/035607; US2004/0167319); the human monoclonal antibodies set forth in WO. 2004/035607 and US2004/0167319 (Teeling et aL); the antibodies having complex N-glycoside-linked sugar chains bound to the Fc region described in US 2004/0093621 (Shitara et al.); monoclonal antibodies and antigen-binding fragments binding to CD20 (WO 2005/000901, Tedder et al.) such as HB20-3, HB20-4, HB20-25, and MB20-1 1; CD20 binding molecules such as the AME series of antibodies, e.g., AME 33 antibodies as set forth in WO 2004/103404 and US2005/0025764 (Watkins et al., Eli Lilly/Applied Molecular Evolution, AME); CD20 binding molecules such as those described in US 2005/0025764 (Watkins et al.); A20 antibody or variants thereof such as chimeric or humanized A20 antibody (cA20, hA20, respectively) (US 2003/0219433, Immunomedics); CD20-binding antibodies, including epitope-depleted Leu-16, 1H4, or 2B8, optionally conjugated with IL-2, as in US 2005/0069545A1 and WO 2005/16969 (Carr et al.); bispecific antibody that binds CD22 and CD20, for example, hLL2xhA20 (WO2005/14618, Chang et al.); monoclonal antibodies L27, G28-2, 93-1B3, B-C1 or NU-B2 available from the International Leukocyte Typing Workshop (Valentine et aL, In: Leukocyte Typing III (McMichael, Ed., p. 440, Oxford University Press (1987)); 1H4 (Haisma et al. Blood 92:184 (1998)). The preferred CD20 antibodies herein are chimeric, humanized, or human CD20 antibodies, more preferably rituximab, humanized 2H7, 2F2 (Hu-Max-CD20) human CD20 antibody (Genmab), and humanized A20 antibody (Immunomedics).

The terms “rituximab” or “RITUXAN®” herein refer to the genetically engineered chimeric murine/human monoclonal antibody directed against the CD20 antigen and designated “C2B8” in U.S. Pat. No. 5,736,137, including fragments thereof which retain the ability to bind CD20.

Purely for the purposes herein and unless indicated otherwise, a “humanized 2H7” antibody is a humanized variant of murine 2H7 antibody, wherein the antibody is effective to reduce circulating B cells in vivo.

In one embodiment, the humanized 2H7 antibody comprises one, two, three, four, five or six of the following CDR sequences:

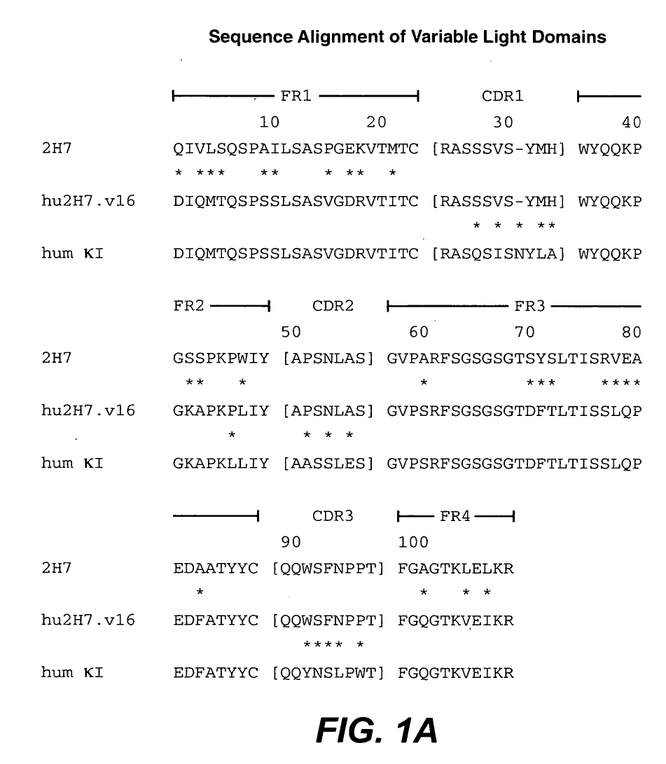

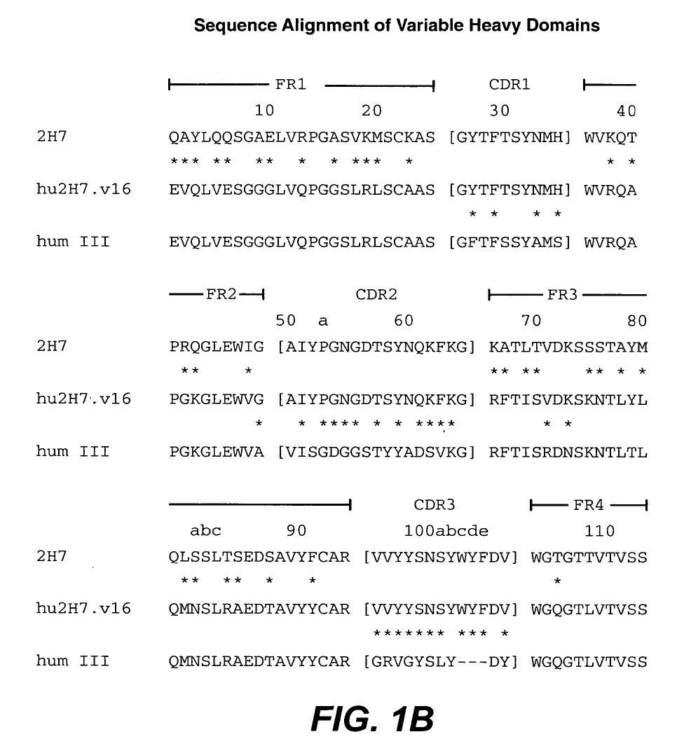

CDR L1 sequence RASSSVSYXH wherein X is M or L (SEQ ID NO. 21), for example SEQ ID NO:4 (FIG. 1A),

CDR L2 sequence of SEQ ID NO: 5 (FIG. 1A),

CDR L3 sequence QQWXFNPPT wherein X is S or A (SEQ ID NO. 22), for example SEQ ID NO: 6 (FIG. 1A),

CDR H1 sequence of SEQ ID NO:10 (FIG. 1B),

CDR H2 sequence of AIYPGNGXTSYNQKFKG wherein X is D or A (SEQ ID NO. 23), for example SEQ ID NO:11 (FIG. 1B), and

CDR H3 sequence of VVYYSXXYWYFDV wherein the X at position 6 is N, A, Y, W or D, and the X as position 7 is S or R (SEQ ID NO. 24), for example SEQ ID NO:12 (FIG. 1B).

The CDR sequences above are generally present within human variable light and variable heavy framework sequences, such as substantially the human consensus FR residues of human light chain kappa subgroup I (VLKI), and substantially the human consensus FR residues of human heavy chain subgroup III (V_{H}III). See also WO 2004/056312 (Lowman et al.).

The variable heavy region may be joined to a human IgG chain constant region, wherein the region may be, for example, IgG1 or IgG3, including native sequence and variant constant regions.

In a preferred embodiment, such antibody comprises the variable heavy domain sequence of SEQ ID NO: 8 (v16, as shown in FIG. 1B), optionally also comprising the variable light domain sequence of SEQ ID NO:2 (v16, as shown in FIG. 1A), which optionally comprises one or more amino acid substitution(s) at positions 56, 100, and/or 100a, e.g. D56A, N100A or N100Y, and/or S100aR in the variable heavy domain and one or more amino acid substitution(s) at positions 32 and/or 92, e.g. M32L and/or S92A, in the variable light domain. Preferably, the antibody is an intact antibody comprising the light chain amino acid sequences of SEQ ID NOs. 13 or 15, and heavy chain amino acid sequences of SEQ ID NO. 14, 16, 17 or 20.

A preferred humanized 2H7 antibody is ocrelizumab (Genentech).

The antibody herein may further comprise at least one amino acid substitution in the Fc region that improves ADCC activity, such as one wherein the amino acid substitutions are at positions 298, 333, and 334, preferably S298A, E333A, and K334A, using Eu numbering of heavy chain residues. See also U.S. Pat. No. 6,737,056B1, Presta.

Any of these antibodies may comprise at least one substitution in the Fc region that improves FcRn binding or serum half-life, for example a substitution at heavy chain position 434, such as N434W. See also U.S. Pat. No. 6,737,056B1, Presta.

Any of these antibodies may further comprise at least one amino acid substitution in the Fc region that increases CDC activity, for example, comprising at least a substitution at position 326, preferably K326A or K326W. See also U.S. Pat. No. 6,528,624B1 (Idusogie et al.).

Some preferred humanized 2H7 variants are those comprising the variable light domain of SEQ ID NO:2 and the variable heavy domain of SEQ ID NO:8, including those with or without substitutions in an Fc region (if present), and those comprising a variable heavy domain with alteration N100; or D56A and N100A; or D56A, N100Y, and S100aR; in SEQ ID NO:8 and a variable light domain with alteration M32L; or S92A; or M32L and S92A; in SEQ ID NO:2.

M34 in the variable heavy domain of 2H7.v16 has been identified as a potential source of antibody stability and is another potential candidate for substitution.

In a summary of some various preferred embodiments of the invention, the variable region of variants based on 2H7.v16 comprise the amino acid sequences of v16 except at the positions of amino acid substitutions that are indicated in Table 3 below. Unless otherwise indicated, the 2H7 variants will have the same light chain as that of v16.

TABLE 3
Exemplary Humanized 2H7 Antibody Variants

| 2H7 | Heavy chain | Light chain | |
|---|---|---|---|
| Version | (V_{H}) changes | (V_{L}) changes | Fc changes |
| 16 for | | | — |
| reference | | | |
| 31 | — | — | S298A, E333A, K334A |
| 73 | N100A | M32L | |
| 75 | N100A | M32L | S298A, E333A, K334A |
| 96 | D56A, N100A | S92A | |
| 114 | D56A, N100A | M32L, S92A | S298A, E333A, K334A |
| 115 | D56A, N100A | M32L, S92A | S298A, E333A, K334A, |
| | | | E356D, M358L |
| 116 | D56A, N100A | M32L, S92A | S298A, K334A, K322A |
| 138 | D56A, N100A | M32L, S92A | S298A, E333A, K334A, |
| | | | K326A |
| 477 | D56A, N100A | M32L, S92A | S298A, E333A, K334A, |
| | | | K326A, N434W |
| 375 | — | — | K334L |
| 588 | — | | S298A, E333A, K334A, |
| | | | K326A |
| 511 | D56A, N100Y, | | S298A, E333A, K334A, |
| | S100aR | | K326A |

One preferred humanized 2H7 comprises 2H7.v16 variable light domain sequence:

(SEQ ID NO:2)

DIQMTQSPSSLSASVGDRVTITCRASSSVSYMHWYQQKPGKAPKLIYAPS
NLASGVPSRFSGSGSGTDFTLTISSLQPEDFATYYCQQWSFNPPTFGQGT
KVEIKR;

and 2H7.v16 variable heavy domain sequence:

(SEQ ID NO:8)

EVQLVESGGGLVQPGGSLRLSCAASGYTFTSYNMHWVRQAPGKGLEWVGA
IYPGNGDTSYNQKFKGRIFTISVDKSKNTLYLQMNSLRAEDTAVYYCARV
VYYSNSYWYFDVWGQGTLVTVSS.

Where the humanized 2H7.v16 antibody is an intact antibody, it may comprise the light chain amino acid sequence:

(SEQ ID NO:13)

DIQMTQSPSSLSASVGDRVTITCRASSSVSYMHWYQQKPGKAPKPLIYAP
SNLASGVPSRFSGSGSGTDFTLTISSLQPEDFATYYCQQWSFNPPTFGQG
TKVEIKRTVAAPSVFIEPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC;

and the heavy chain amino acid sequence of SEQ ID NO. 14 or:

(SEQ ID NO:17)
EVQLVESGGGLVQPGGSLRLSCAASGYTFTSYNMHWVRQAPGKGLEWVGA
IYPGNGDTSYNQKFKGRFTISVDKSKNTLYLQMNSLRAEDTAVYYCARVV
YYSNSYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPLEKTISKAKGQPRE
PQVYTLPPSREEMTKNQVSLTCLVKGFYPSDLAVEWESNGQPENNYKTTP
PVLDSDGSFFLYSKLTVDKSRWQQGNVFSCSVMHEALHNHYTQK
SLSLSPG.

Another preferred humanized 2H7 antibody comprises 2H7.v5 11 variable light domain sequence:

(SEQ ID NO:18)

DIQMTQSPSSLSASVGDRVTITCRASSSVSYLHWYQQKPGKAPKPLIYAP
SNLASGVPSRESGSGSGTDFTLTISSLQPEDFATYYCQQWAFNPPTFGQG
TKVEIKR

and 2H7.v511 variable heavy domain sequence:

(SEQ ID NO:19)

EVQLVESGGGLVQPGGSLRLSCAASGYTFTSYNMHWVRQAPGKGLEWVGA
IYPGNGATSYNQKFKGRETISVDKSKNTLYLQMNSLRAEDTAVYYCARVV
YYSYRYWYFDVWGQGTLVTVSS.

Where the humanized 2H7.v511 antibody is an intact antibody, it may comprise the light chain amino acid sequence:

(SEQ ID NO:15)

DIQMTQSPSSLSASVGDRVTITCRASSSVSYLHWYQQKPGKAPKPLIYAP
SNLASGVPSRFSGSGSGTDFTLTISSLQPEDFATYYCQQWAFNPPTFGQG
TKVELKRTVAAPSVFTFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC

and the heavy chain amino acid sequence of SEQ ID NO. 16 or:

(SEQ ID NO:20)

EVQLVESGGGLVQPGGSLRLSCAASGYTFTSYNMHWVRQAPGKGLEWVGA
IYPGNGATSYNQKFKGRFTISVDKSKNTLYLQMNSLRAEDTAVYYCARVV
YYSYRYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNATYRVVSVLTVLHQDWLNGKEYKCKVSNAALPAPLAATISKAKGQPRE
PQVYTLPPSREEMTKISIQVSLTCLVKGFYPSDIAVEWESNGQPENNYKT
TPPVLDSDGSFFLYSKLTVDKSRWQQGNVFSCSVMHEALHNHYTQ
KSLSLSPG.

“Growth-inhibitory” antibodies are those that prevent or reduce proliferation of a cell expressing an antigen to which the antibody binds. For example, the antibody may prevent or reduce proliferation of B cells in vitro and/or in vivo.

Antibodies that “induce apoptosis” are those that induce programmed cell death, e.g. of a B cell, as determined by standard apoptosis assays, such as binding of annexin V, fragmentation of DNA, cell shrinkage, dilation of endoplasmic reticulum, cell fragmentation, and/or formation of membrane vesicles (called apoptotic bodies).

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the P-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-binding sites and is still capable of cross-linking antigen.

“Fv” is the minimum antibody fragment that contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)₂ antibody fragments originally were produced as pairs of Fab′ fragments that have hinge cysteines between them. Other chemical couplings of antibody fragments are also known.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their “heavy chains,” (if present) antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2. The heavy chain constant domains that correspond to the different classes of antibodies are called α, δ, ε, γ, and μ, respectively. The subunit structures and three-dimensional configurations of different classes of immunoglobulins are well known.

Unless indicated otherwise, herein the numbering of the residues in an immunoglobulin heavy chain is that of the EU index as in Kabat et al., Sequences of Proteins of Immunological Interest, 5th Ed. Public Health Service, National Institutes of Health, Bethesda, Md. (1991), expressly incorporated herein by reference. The “EU index as in Kabat” refers to the residue numbering of the human IgG1 EU antibody.

The term “Fc region” herein is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue.

A “functional Fc region” possesses an “effector function” of a native sequence Fc region. Exemplary “effector functions” include C1q binding; complement dependent cytotoxicity; Fc receptor binding; antibody-dependent cell-mediated cytotoxicity (ADCC); phagocytosis; down regulation of cell surface receptors (e.g. B cell receptor; BCR), etc. Such effector functions generally require the Fc region to be combined with a binding domain (e.g. an antibody variable domain) and can be assessed using various assays as herein disclosed, for example.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region; as well as naturally occurring variants of any of the above.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith.

“Antibody-dependent cell-mediated cytotoxicity” and “ADCC” refer to a cell-mediated reaction in which nonspecific cytotoxic cells that express Fc receptors (FcRs) (e.g. Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcR expression on hematopoietic cells in summarized is Table 3 on page 464 of Ravetch and Kinet, Annu. Rev. Immunol. 9:457-492 (1991). To assess ADCC activity of a molecule of interest, an in vitro ADCC assay, such as that described in U.S. Pat. Nos. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1998).

“Human effector cells” are leukocytes that express one or more FcRs and perform effector functions. Preferably, the cells express at least FcγRII and carry out ADCC effector function. Examples of human leukocytes that mediate ADCC include peripheral blood mononuclear cells (PBMC), natural killer (NK) cells, monocytes, cytotoxic T cells and neutrophils; with PBMCs and NK cells being preferred.

The terms “Fc receptor” or “FcR” are used to describe a receptor that binds to the Fc region of an antibody. The preferred FcR is a native-sequence human FcR. Moreover, a preferred FcR is one that binds an IgG antibody (a gamma receptor) and includes receptors of the FcγRI, FcγRII, and Fcγ RIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see Daeron, Annu. Rev. ImmunoL 15:203-234 (1997)). FcRs are reviewed in Ravetch and Kinet, Annu. Rev. Immunol 9:457-492 (1991); Capel et al., Immunomethods 4:25-34 (1994); and de Haas et al., J. Lab. Clin. Med. 126:330-341 (1995). Other FcRs, including those to be identified in the future, are encompassed by the term “FcR” herein. The term also includes the neonatal receptor, FcRn, which is responsible for the transfer of maternal IgGs to the fetus and immunoglobulin homeostasis (Guyer et al., J. Immunol. 117:587 (1976) and Kim et al, J. Immunol. 24:249 (1994)).

“Complement dependent cytotoxicity” or “CDC” refers to the ability of a molecule to lyse a target in the presence of complement. The complement activation pathway is initiated by the binding of the first component of the complement system (Clq) to a molecule (e.g. an antibody) complexed with a cognate antigen. To assess complement activation, a CDC assay, e.g. as described in Gazzano-Santoro et al., J. Immunol. Methods 202:163 (1996), may be performed.

“Single-chain Fv” or “scFv” antibody fragments comprise the V_{H} and V_{L} domains of antibody, wherein these domains are present in a single polypeptide chain. Preferably, the Fv polypeptide further comprises a polypeptide linker between the V_{H} and V_{L} domains that enables the scFv to form the desired structure for antigen binding. For a review of scFv see Plückthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

The term “monoclonal antibody” as used herein refers to an antibody from a population of substantially homogeneous antibodies, i.e., the individual antibodies comprising the population are identical and/or bind the same epitope(s), except for possible variants that may arise during production of the monoclonal antibody, such variants generally being present in minor amounts. Such monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones or recombinant DNA clones. It should be understood that the selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations which typically include different antibodies directed against different determinants (epitopes), each monoclonal antibody of a monoclonal antibody preparation is directed against a single determinant on an antigen. In addition to their specificity, the monoclonal antibody preparations are advantageous in that they are typically uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler et al., Nature, 256:495 (1975); Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681, (Elsevier, N.Y., 1981)), recombinant DNA methods (see, e.g., U.S. Pat. No. 4,816,567), phage display technologies (see, e.g., Clackson et al., Nature, 352:624-628 (1991); Marks etal., J. Mol. Biol., 222:581-597 (1991); Sidhu etal., J. Mol. Biol. 338(2):299-310 (2004); Lee et al., J. Mol. Biol.340(5):1073-1093 (2004); Fellouse, Proc. Nat. Acad. Sci. USA 101(34):12467-12472 (2004); and Lee et al. J. Immunol. Methods 284(1-2):119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA, 90:2551 (1993); Jakobovits et al., Nature, 362:255-258 (1993); Bruggemann et al., Year in Immuno., 7:33 (1993); U.S. Pat. Nos. 5,545,806; 5,569,825; 5,591,669 (all of GenPharm); 5,545,807; WO 1997/17852; U.S. Pat. Nos. 5,545,807; 5,545,806; 5,569,825; 5,625,126; 5,633,425; and 5,661,016; Marks et al., Bio/Technology, 10: 779-783 (1992); Lonberg et al., Nature, 368: 856-859 (1994); Morrison, Nature, 368: 812-813 (1994); Fishwild et al., Nature Biotechnology, 14: 845-851 (1996); Neuberger, Nature Biotechnology, 14: 826 (1996); and Lonberg and Huszar, Intern. Rev. ImmunoL, 13: 65-93 (1995).

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, such as baboon, rhesus or cynomolgus monkey) and human constant region sequences (U.S. Pat. No. 5,693,780).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence, except for FR substitution(s) as noted above. The humanized antibody optionally also will comprise at least a portion of an immunoglobulin constant region, typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

The term “hypervariable region” when used herein refers to the amino acid residues of an antibody that are responsible for antigen binding. The hypervariable region comprises amino acid residues from a “complementarity determining region” or “CDR” (e.g. residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). “Framework” or “FR” residues are those variable domain residues other than the hypervariable region residues as herein defined.

A “naked antibody” is an antibody (as herein defined) that is not conjugated to a heterologous molecule, such as a cytotoxic moiety or radiolabel.

An “intact antibody” herein is one which comprises two antigen binding regions, and an Fc region. Preferably, the intact antibody has a functional Fc region.

An “isolated” antibody is one that has been identified and separated and/or recovered from a component of its natural environment. Contaminant components of its natural environment are materials that would interfere with diagnostic or therapeutic uses for the antibody, and may include enzymes, hormones, and other proteinaceous or non-proteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

An “affinity matured” antibody is one with one or more alterations in one or more hypervariable regions thereof which result an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures known in the art. Marks et al. Bio/Technology 10:779-783 (1992) describes affinity maturation by V_{H} and V_{L} domain shuffling. Random mutagenesis of CDR and/or framework residues is described by: Barbas et al. Proc Nat. Acad. Sci, USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

“Treatment” of a subject herein refers to both therapeutic treatment and prophylactic or preventative measures. Those in need of treatment include those already with a IBD as well as those in which the IBD is to be prevented. Hence, the subject may have been diagnosed as having the IBD or may be predisposed or susceptible to the IBD. The term “treating”, “treat” or “treatment” as used herein includes preventative (e.g., prophylactic), palliative and curative treatment.

The term “immunosuppressive agent” as used herein for adjunct therapy refers to substances that act to suppress or mask the immune system of the subject being treated herein. This would include substances that suppress cytokine production, down-regulate or suppress self-antigen expression, or mask the MHC antigens. Examples of such agents include 2-amino-6-aryl-5-substituted pyrimidines (see U.S. Pat. No. 4,665,077); non-steroidal anti-inflammatory drugs (NSAIDs); ganciclovir; tacrolimus; glucocorticoids such as cortisol or aldosterone; anti-inflammatory agents such as a cyclooxygenase inhibitor; a 5-lipoxygenase inhibitor; or a leukotriene receptor antagonist; purine antagonists such as azathioprine or mycophenolate mofetil (MMF); alkylating agents such as cyclophosphamide; bromocryptine; danazol; dapsone; glutaraldehyde (which masks the MHC antigens, as described in U.S. Pat. No. 4,120,649); anti-idiotypic antibodies for MHC antigens and MHC fragments; cyclosporine; 6 mercaptopurine; steroids such as corticosteroids or glucocorticosteroids or glucocorticoid analogs, e.g., prednisone, methylprednisolone, including SOLU-MEDROL® methylprednisolone sodium succinate, and dexamethasone; dihydrofolate reductase inhibitors such as methotrexate (oral or subcutaneous); anti-malarial agents such as chloroquine and hydroxychloroquine; sulfasalazine; leflunomide; cytokine or cytokine receptor antibodies or antagonists including anti-interferon-alpha, -beta, or -gamma antibodies, anti-tumor necrosis factor(TNF)-alpha antibodies (infliximab (REMICADE®) or adalimumab), anti-TNF-alpha immunoadhesin (etanercept), anti-TNF-beta antibodies, anti-interleukin-2 (IL-2) antibodies and anti-IL-2 receptor antibodies, and anti-interleukin-6 (IL-6) receptor antibodies and antagonists; anti-LFA-1 antibodies, including anti-CD11a and anti-CD18 antibodies; anti-L3T4 antibodies; heterologous anti-lymphocyte globulin; pan-T antibodies, preferably anti-CD3 or anti-CD4/CD4a antibodies; soluble peptide containing a LFA-3 binding domain (WO 90/08187 published Jul. 26, 1990); streptokinase; transforming growth factor-beta (TGF-beta); streptodomase; RNA or DNA from the host; FK506; RS-61443; chlorambucil; deoxyspergualin; rapamycin; T-cell receptor (Cohen et al., U.S. Pat. No. 5,114,721); T-cell receptor fragments (Offner et al., Science, 251: 430-432 (1991); WO 90/11294; Ianeway, Nature, 341: 482 (1989); and WO 91/01133); BAFF antagonists such as BAFF or BR3 antibodies or immunoadhesins and zTNF4 antagonists (for review, see Mackay and Mackay, Trends Immunol., 23:113-5 (2002) and see also definition below); biologic agents that interfere with T cell helper signals, such as anti-CD40 receptor or anti-CD40 ligand (CD154), including blocking antibodies to CD40-CD40 ligand.(e.g., Durie etal., Science, 261: 1328-30 (1993); Mohan etal., J. Immunol., 154: 1470-80 (1995)) and CTLA4-Ig (Finck et al., Science, 265: 1225-7 (1994)); and T-cell receptor antibodies (EP 340,109) such as T10B9.

The term “cytotoxic agent” as used herein refers to a substance that inhibits or prevents the function of cells and/or causes destruction of cells. The term is intended to include radioactive isotopes (e.g. At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents, and toxins such as small-molecule toxins or enzymatically active toxins of bacterial, fungal, plant or animal origin, or fragments thereof.

A “chemotherapeutic agent” is a chemical compound useful in the treatment of cancer. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and. bullatacinone); delta-9-tetrahydrocannabinol (dronabinol, MARINOL(®); beta-lapachone; lapachol; colchicines; betulinic acid; a camptothecin (including the synthetic analogue topotecan (HYCAMTIN®), CPT-11 (irinotecan, CAMPTOSAR(®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e. g., calicheamicin, especially calicheamicin gamma1I and calicheamicin omegaIl (see, e.g., Agnew, Chem Intl. Ed. Engl., 33: 183-186 (1994)); dynemicin, including dynemicin A; an esperamicin; as well as neocarzinostatin chromophore and related chromoprotein enediyne antiobiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®) and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene; Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., paclitaxel (TAXOL®), albumin-engineered nanoparticle formulation of paclitaxel (ABRAXANE™), and doxetaxel (TAXOTERE®); chloranbucil; 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluoromethylornithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin.

Also included in this definition are anti-hormonal agents that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX® tamoxifen), raloxifene (EVISTA®), droloxifene, 4-hydroxytamoxifen, trioxifene, keoxifene, LY117018, onapristone, and toremifene (FARESTON®); anti-progesterones; estrogen receptor down-regulators (ERDs); estrogen receptor antagonists such as fulvestrant (FASLODEX®); agents that function to suppress or shut down the ovaries, for example, leutinizing hormone-releasing hormone (LHRH) agonists such as leuprolide acetate (LUPRON® and ELIGARD®), goserelin acetate, buserelin acetate and tripterelin; anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, megestrol acetate (MEGASE®), exemestane (AROMASIN®), formestanie, fadrozole, vorozole (RIVISOR®), letrozole (FEMARA®), and anastrozole (ARIMIDEX®). In addition, such definition of chemotherapeutic agents includes bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); as well as troxacitabine (a 1,3-dioxolane nucleoside cytosine analog); antisense oligonucleotides, particularly those that inhibit expression of genes in signaling pathways implicated in abherant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); lapatinib ditosylate (an ErbB-2 and EGFR dual tyrosine kinase small-molecule inhibitor also known as GW572016); and pharmaceutically acceptable salts, acids or derivatives of any of the above.

The term “cytokine” is a generic term for proteins released by one cell population that act on another cell as intercellular mediators. Examples of such cytokines are lymphokines, monokines; interleukins (ILs) such as IL-1, IL-1α, IL-2, IL-3, IL-4, IL-5, IL-6, IL-7, IL-8, IL-9, IL-11, IL-12, IL-15, including PROLEUKIN® IL-2 and human IL-4 and mutants of human IL4, such as, for example, a mutant containing a mutation in the region of IL-4 which is involved in binding to IL-2R gamma, e.g., Arg 21 is changed to a Glu residue; a tumor necrosis factor such as TNF-α or TNF-β; and other polypeptide factors including LIF and kit ligand (KL). As used herein, the term cytokine includes proteins from natural sources or from recombinant cell culture and biologically active equivalents of the native-sequence cytokines, including synthetically produced small-molecule entities and pharmaceutically acceptable derivatives and salts thereof.

The term “hormone” refers to polypeptide hormones, which are generally secreted by glandular organs with ducts. Included among the hormones are, for example, growth hormone such as human growth hormone, N-methionyl human growth hormone, and bovine growth hormone; parathyroid hormone; thyroxine; insulin; proinsulin; relaxin; estradiol; hormone-replacement therapy; androgens such as calusterone, dromostanolone propionate, epitiostanol, mepitiostane, or testolactone; prorelaxin; glycoprotein hormones such as follicle stimulating hormone (FSH), thyroid stimulating hormone (TSH), and luteinizing hormone (LH); prolactin, placental lactogen, mouse gonadotropin-associated peptide, gonadotropin-releasing hormone; inhibin; activin; mullerian-inhibiting substance; and thrombopoietin. As used herein, the term hormone includes proteins from natural sources or from recombinant cell culture and biologically active equivalents of the native-sequence hormone, including synthetically produced small-molecule entities and pharmaceutically acceptable derivatives and salts thereof.

The term “growth factor” refers to proteins that promote growth, and include, for example, hepatic growth factor; fibroblast growth factor; vascular endothelial growth factor; nerve growth factors such as NGF-β; platelet-derived growth factor; transforming growth factors (TGFs) such as TGF-α and TGF-β; insulin-like growth factor-I and -II; erythropoietin (EPO); osteoinductive factors; interferons such as interferon-α, -β, and -γ; and colony stimulating factors (CSFs) such as macrophage-CSF (M-CSF); granulocyte-macrophage-CSF (GM-CSF); and granulocyte-CSF (G-CSF). As used herein, the term growth factor includes proteins from natural sources or from recombinant cell culture and biologically active equivalents of the native-sequence growth factor, including synthetically produced small-molecule entities and pharmaceutically acceptable derivatives and salts thereof.

The term “integrin” refers to a receptor protein that allows cells both to bind to and to respond to the extracellular matrix and is involved in a variety of cellular functions such as wound healing, cell differentiation, homing of tumor cells and apoptosis. They are part of a large family of cell adhesion receptors that are involved in cell-extracellular matrix and cell-cell interactions. Functional integrins consist of two transmembrane glycoprotein subunits, called alpha and beta, that are non-covalently bound. The alpha subunits all share some homology to each other, as do the beta subunits. The receptors always contain one alpha chain and one beta chain. Examples include Alpha6beta1, Alpha3beta1, Alpha7beta1, LFA-1 etc. As used herein, the term “integrin” includes proteins from natural sources or from recombinant cell culture and biologically active equivalents of the native-sequence integrin, including synthetically produced small-molecule entities and pharmaceutically acceptable derivatives and salts thereof.

For the purposes herein, “tumor necrosis factor alpha (TNF-alpha)” refers to a human TNF-alpha molecule comprising the amino acid sequence as described in Pennica et al., Nature, 312:721 (1984) or Aggarwal et al., JBC, 260:2345 (1985). A “TNF-alpha inhibitor” herein is an agent that inhibits, to some extent, a biological function of TNF-alpha, generally through binding to TNF-alpha and neutralizing its activity. Examples of TNF inhibitors specifically contemplated herein are etanercept (ENBREL®), infliximab (REMICADE®), and adalimumab (HUMIRA™).

Examples of “disease-modifying anti-rheumatic drugs” or “DMARDs” include hydroxycloroquine, sulfasalazine, methotrexate, leflunomide, etanercept, infliximab, azathioprine, D-penicillamine, gold salts (oral), gold salts (intramuscular), minocycline, cyclosporine including cyclosporine A and topical cyclosporine, staphylococcal protein A (Goodyear and Silverman, J. Exp. Med., 197, (9), p 1125-39 (2003)), including salts and derivatives thereof, etc.

Examples of “non-steroidal anti-inflammatory drugs” or “NSAIDs” include aspirin, acetylsalicylic acid, ibuprofen, naproxen, indomethacin, sulindac, tolmetin, COX-2 inhibitors such as celecoxib (CELEBREX®; 4-(5-(4-methylphenyl)-3-(trifluoromethyl)-1H-pyrazol-1-yl) benzenesulfonamide and valdecoxib (BEXTRA(®), and meloxicam (MOBIC®), including salts and derivatives thereof, etc.

Examples of “integrin antagonists or antibodies” herein include an LFA-1 antibody, such as efalizumab (RAPTIVA®) commercially available from Genentech, or an alpha 4 integrin antibody such as natalizumab (ANTEGREN®) available from Biogen, or diazacyclic phenylalanine derivatives (WO 2003/89410), phenylalanine derivatives (WO 2003/70709, WO 2002/28830, WO 2002/16329 and WO 2003/53926), phenylpropionic acid derivatives (WO 2003/10135), enamine derivatives (WO 2001/79173), propanoic acid derivatives (WO 2000/37444), alkanoic acid derivatives (WO 2000/32575), substituted phenyl derivatives (US Pat. Nos. 6,677,339 and 6,348,463), aromatic amine derivatives (U.S. Pat. No. 6,369,229), ADAM disintegrin domain polypeptides (US2002/0042368), antibodies to alphavbeta3. integrin (EP 633945), aza-bridged bicyclic amino acid derivatives (WO 2002/02556), etc.

“Corticosteroid” refers to any one of several synthetic or naturally occurring substances with the general chemical structure of steroids that mimic or augment the effects of the naturally occurring corticosteroids. Examples of synthetic corticosteroids include prednisone, prednisolone (including methylprednisolone, such as SOLU-MEDROL® methylprednisolone sodium succinate), dexamethasone or dexamethasone triamcinolone, hydrocortisone, and betamethasone. The preferred corticosteroids herein are prednisone, methylprednisolone, hydrocortisone, or dexamethasone.

As used herein, the term “effective amount” is meant to refer to an amount of the antibody or antagonist that is effective for treating the IBD. Effective amounts are typically determined by the effect they have compared to the effect observed when a composition that includes no active ingredient (ie. a control) is administered to a similarly situated individual.

A “package insert” is used to refer to instructions customarily included in commercial packages of therapeutic products, that contain information about the indications, usage, dosage, administration, contraindications, other therapeutic products to be combined with the packaged product, and/or warnings concerning the use of such therapeutic products, etc.

A “medicament” is an active drug to treat the IBD or its symptoms or side effects.

II. Therapy of IBD

The invention herein provides a method for treating IBD in a human subject comprising administering to the subject an effective amount of an antibody (or antagonist) that binds to a B-cell surface marker, such as CD20.

In particular, the invention provides a method for treating moderate-severe inflammatory bowel disease (IBD) in a human subject comprising administering to the subject an effective amount of a CD20 antibody (or antagonist), wherein administration of the antibody (or antagonist) results in a clinical response and/or disease remission.

Such administration may also reduce B-cells in colonic mucosa, Peyer's patches, secondary lymphoid tissues or organs such as the lymph nodes and spleen, and blood, but especially the colonic mucosa of the subject.

The IBD may be ulcerative colitis (UC), or Crohn's disease, but preferably UC. The subject treated herein may have active IBD, active UC or active Crohn's disease. Generally, the subject treated will have moderate-severe IBD, moderate-severe UC, or moderate-severe Crohn's disease.

Moreover, the subject may have steroid-refractory and/or steroid dependent IBD, steroid-refractory and/or steroid dependent UC or steroid-refractory and/or steroid dependent Crohn's disease.

Subjects treated herein may: have had diagnosis of IBD ≧6 months at screening; have ≧20 cm of active disease at screening sigmoidoscopy; have active disease as defined by a DAI score between ≧6 and ≦11, with ≧2 for rectal bleeding and ≧2 for flexible sigmoidoscopy; have been treated with oral corticosteroids for UC within 2 years prior to screening; have been treated with an intensity greater than a prednisone equivalent dose of 20 mg/day for at least 2 weeks' duration; be resistant or refractory to etanercept, infliximab, or adalimumab; have been treated with a stable doses of aminosalicylate for ≧3 weeks; have been treated with stable doses of oral corticosteroid dose for ≧2 weeks; have been treated with 6-MP for a 3-month period, and with a stable dose thereof for ≧4 weeks; have been treated with azathioprine for a 3-month period, with a stable dose for ≧4 weeks.

The standard of care for subjects with active moderate-severe active UC involves therapy with standard doses of: an aminosalicylate, an oral corticosteroid, 6-mercaptopurine (6-MP) and/or azathioprine. Therapy with a CD20 antibody as disclosed herein will result in an improvement in disease remission (rapid control of disease and/or prolonged remission), and/or clinical response, superior to that achieved with the standard of care for such subjects.

Administration of the antibody may result in disease remission, for example where disease remission is achieved at, or by, about week 8. Preferably, the time to disease remission is less than that achieved in a subject who is not treated with the CD20 antibody. Moreover, preferably the duration of remission is greater than that achieved in a subject who is not treated with the CD20 antibody. For example, the duration of remission may be for at least 24 weeks, and preferably for at least 48 weeks, and most preferably for at least about 2 years, from initial treatment or from achievement of remission. Remission may be defined as a sigmoidoscopy score of 0 or 1, and/or rectal bleeding score of 0.

Administration of the antibody may result in a clinical response, for example where the clinical response is achieved at, or by, about week 8. Clinical response herein may be defined as a reduction in disease activity index (DAI) score, for example, reduction of such score by greater than or equal to 3 points.

In one embodiment, the subject has never been previously treated with a CD20 antibody. Preferably, the subject is not suffering from a B cell malignancy. The subject is also preferably one who is not suffering from an autoimmune disease, other than IBD, UC, or Crohn's disease.

Also provided, is a method for reducing a disease activity index (DAI) score in a human subject with active ulcerative colitis (UC) comprising administering a CD20 antibody to the subject in an amount effective to reduce DAI score. Preferably, the DAI scoring system is as in Table 2 herein, and administration of the CD20 antibody reduces such DAI score by greater than or equal to 3 points.

An addition, the method involves treatment of active inflammatory bowel disease (IBD) in a human subject with atypical perinuclear antineutrophil cytoplasmic antibody (p-ANCA) and/or anti-human tropomyosin isoform 5 (hTM5) autoantibody level(s). Administration of a CD20 antibody to the subject effectively reduces the p-ANCA and/or anti-hTM5 antibody level(s) in the subject.

The exact dose will be determined by the clinician according to accepted standards, taking into account the nature and severity of the condition to be treated, the type of antagonist or antibody, the subject's traits, etc. Determination of dose is within the level of ordinary skill in the art. Preferably the antibody is administered systemically, intravenously, or subcutaneously. Depending upon the route and method of administration, the antagonist or antibody may be administered in a single dose, as a prolonged infusion, or intermittently over an extended period. Intravenous administration will generally be by bolus injection or infusion over a typical period of one to several hours. Sustained release formulations can be employed.

In a preferred embodiment, the method comprises administering one or more doses in the range from about 200 mg to 2000 mg, preferably about 500 mg to 1500 mg, and most preferably about 750 mg to 1200 mg. For example, one to four doses, or only one or two doses may be administered. According to this embodiment, the antibody may be administered within a period of about one month, preferably within a period of about 2 to 3 weeks, and most preferably within a period of about two weeks.

Where more than one dose is administered, the later dose (for example, second or third dose) is preferably administered from about 1 to 20 days, more preferably from about 6 to 16 days, and most preferably from about 14 to 16 days from the time the previous dose was administered. The separate doses are preferably administered within a total period of between about 1 day and 4 weeks, more preferably between about I and 20 days (e.g., within a period of 6-18 days). Each such separate dose of the antibody is preferably about 200 mg to 2000 mg, preferably about 500 mg to 1500 mg, and most preferably about 750 mg to 1200 mg.

As noted above, however, these suggested amounts of antagonist or antibody are subject to a great deal of therapeutic discretion. The key factor in selecting an appropriate dose and scheduling is the result obtained, as indicated above. For example, relatively higher doses may be needed initially for the treatment of active IBD. A subsequent dose may be higher than an earlier dose. To obtain the most efficacious results, the antagonist or antibody is generally administered as close to the first sign, diagnosis, appearance, or occurrence of the disease or disorder as possible or during remissions of the disease or disorder.

Hence, the invention provides a method for treating inflammatory bowel disease (IBD) in a human subject with active IBD comprising administering only one or two doses of a CD20 antibody to the subject, wherein disease remission or clinical response is achieved upon administration of the one or two doses of the CD20 antibody. Preferably such one or two doses are administered intravenously (IV), or subcutaneously (SQ). Where two intravenous doses are administered, preferably each of the two doses is in the range from about 200 mg to about 2000 mg.

The antagonist or antibody is administered by any suitable means, including parenteral, subcutaneous, intra-peritoneal, inhalational, intrathecal, intra-articular, and intra-nasal, and, if desired for local immunosuppressive treatment, intralesional administration. Parenteral infusions include, intramuscular, intravenous, intra-arterial, intraperitoneal, or subcutaneous administration. In addition, the antagonist or antibody may suitably be administered by pulse infusion, e.g., with declining doses of the antagonist or antibody. Preferably the dosing is given by injections, most preferably intravenous or subcutaneous injections, depending in part on whether the administration is brief or chronic:

The subject may be retreated with the antagonist or antibody, as by being given more than one exposure or set of doses, such as at least about two exposures of the antagonist or antibody, for example, from about 2 to 60 exposures, and more particularly about 2 to 40 exposures, most particularly, about 2 to 20 exposures.

In one embodiment, any retreatment may be given when signs or symptoms of disease return, when the subject is no longer in remission, and/or when p-ANCA or anti-hTM5 autoantibody levels rise, etc.

In another embodiment, any retreatment may be given at defined intervals. For example, subsequent exposures may be administered at various intervals, such as, for example, about 24-28 weeks or 48-56 weeks or longer. Preferably, such exposures are administered at intervals each of about 24-26 weeks or about 38-42 weeks, or about 50-54 weeks.

In one embodiment, each antagonist or antibody exposure is provided as a single dose of the antagonist or antibody. In an alternative embodiment, each antagonist or antibody exposure is provided as separate doses of the antibody. However, not every antagonist or antibody exposure need be provided as a single dose or as separate doses.

The preferred antagonist is an antibody. In the methods set forth herein, the CD20 antibody may be a naked antibody or may be conjugated with another molecule such as a cytotoxic agent or cytokine. Preferably, the antibody is an intact, naked antibody. The preferred CD20 antibody herein is a chimeric, humanized, or human CD20 antibody, more preferably rituximab, humanized 2H7, 2F2 (HuMax-CD20) human CD20 antibody (Genmab), humanized A20 antibody (Immunomedics). Still more preferred is rituximab or humanized 2H7.

In a further embodiment of all the methods herein, the subject has never been previously treated with drug(s), such as an agent that treats IBD and/or has never been previously treated with an antagonist or antibody to a B-cell surface marker (e.g. has never been previously treated with a CD20 antibody).

In any of the methods herein, one may administer to the subject along with the antagonist or antibody that binds a B-cell surface marker an effective amount of a second medicament (where the antagonist or antibody that binds a B-cell surface marker (e.g., the CD20 antibody) is a first medicament). The type of such second medicament depends on various factors, including the type of IBD, the severity of the IBD, the condition and age of the subject, the type and dose of first medicament employed, etc.

Examples of such additional medicaments or other therapies include another agent that treats IBD, a chemotherapeutic agent, an interferon class drug such as interferon-alpha (e.g., from Amarillo Biosciences, Inc.), IFN-beta-1a (REBIF® and AVONEX®) or IFN-beta-1b (BETASERON®), an oligopeptide such as glatiramer acetate (COPAXONE®), an agent blocking CD40-CD40 ligand, a cytotoxic agent (such as mitoxantrone (NOVANTRONE®), methotrexate, cyclophosphamide; chlorambucil, leflunomide, and azathioprine), one or more immunosuppressive agents (e.g. azathioprine, 6-mercaptopurine, cyclosporine), intravenous immunoglobulin (gamma globulin), lymphocyte-depleting therapy (e.g., mitoxantrone, cyclophosphamide, CAMPATH™ antibodies, anti-CD4, cladribine), a polypeptide construct with at least two domains comprising a de-immunized, autoreactive antigen or its fragment that is specifically recognized by the Ig receptors of autoreactive B-cells (WO 2003/68822), total body irradiation, bone marrow transplantation, integrin antagonist or antibody (e.g., an LFA-1 antibody such as efalizumab (RAPTIVA®) commercially available from Genentech, or an alpha 4 integrin antibody such as natalizumab (ANTEGREN®) available from Biogen Idec, or others as noted above), steroid such as corticosteroid (e.g., methylprednisolone such as SOLUMEDROL™ methylprednisolone sodium succinate for injection, prednisone such as low-dose prednisone, dexamethasone, or glucocorticoid, including systemic corticosteroid therapy), non-lymphocyte-depleting immunosuppressive therapy (e.g., MMF or cyclosporine), cholesterol-lowering drug of the “statin” class (which includes cerivastatin (BAYCOL™), fluvastatin (LESCOL™), atorvastatin (LIPITOR™), lovastatin (MEVACOR™), pravastatin (PRAVACHOL™), and simvastatin (ZOCOR™)), estradiol, testosterone (optionally at elevated dosages; Stuve et al. Neurology 8:290-301 (2002)), androgen, hormone-replacement therapy, a TNF inhibitor such as etanercept (ENBREL®), infliximab (REMICADE®), and adalimumab (HUMIRA™), disease-modifying anti-rheumatic drug (DMARD), nonsteroidal anti-inflammatory drug (NSAID), plasmapheresis or plasma exchange, trimethoprim-sulfamethoxazole (BACTRIM™, SEPTRA™), mycophenolate mofetil, H2-blockers or proton-pump inhibitors (during the use of potentially ulcerogenic immunosuppressive therapy), levothyroxine, cyclosporin A (e.g. SANDIMMUNE®), somatastatin analogue, cytokine, cytokine or cytokine receptor antibody or antagonist, anti-metabolite, rehabilitative surgery or colectomy, radioiodine, thyroidectomy, BAFF antagonist such as BAFF or BR3 antibodies or iinmunoadhesins, anti-CD40 receptor or anti-CD40 ligand (CD154), anti-IL-6 receptor antagonist or antibody, anti-IL-2 antibody such as daclizumab, another B-cell surface antagonist or antibody such as a humanized 2H7 or other humanized or human CD20 antibody with rituximab, oral corticosteroids (e.g. within 2 years prior to initial treatment with the CD20 antibody or antagonist), prednisone (e.g. prednisone equivalent dose of 20 mg/day for at least 2 weeks' duration), etanercept, infliximab, adalimumab, aminosalicylate (e.g. stable dose for ≧3 weeks), oral corticosteroids (e.g. stable dose for ≧2 weeks), 6-MP (e.g. treatment for a 3-month period, with a stable dose for ≧4 weeks), azathioprine (e.g. treatment for a 3-month period, with a stable dose for ≧4 weeks), calcineurin inhibitor, cyclosporine, tacrolimus, sirolimus, methotrexate, mycophenolate mofetil, topical rectal preparation, non-biologic cell-depleting therapy such as ADACOLUMN®, antibiotic, antidiarrheal, bile-acid binder such as cholestyramine, oral and/or topical 5-ASA, oral and/or topical steroid, MLN-02, mesalamine, cortisone cream, hydrocortisone enema, sulfasalazine, alsalazine, balsalazide, methylprednisolone, hydrocortisone, ACTH, intravenous corticosteroids, GELTEX™ (Genzyme), anti-CD3 antibody such as visilizumab (NUVION®), OPC-6535, CBP 1011, thalidomide, ISIS 2302, BXT-51072, a growth factor such as keratinocyte growth factor-2 (KGF-2; REPIFERMIN™), RPD-58, antegren, FK-506, etc.

Preferred second medicaments include one, two, three or four of: an aminosalicylate, an oral corticosteroid, 6-mercaptopurine (6-MP) and azathioprine.

In one preferred method of “combination therapy,” herein, the invention concerns a method for treating inflammatory bowel disease (IBD) in a human subject with active IBD comprising administering to the subject an effective amount of a CD20 antibody and further comprising administering to the subject an effective amount of a second medicament selected from the group consisting of an aminosalicylate, an oral corticosteroid, 6-mercaptopurine (6-MP) and azathioprine.

All these second medicaments may be used in combination with each other or by themselves with the first medicament, so that the expression “second medicament” as used herein does not mean it is the only medicament besides the first medicament, respectively. Thus, the second medicament need not be one medicament, but may constitute or comprise more than one such drug.

These second medicaments as set forth herein are generally used in. the same dosages and with administration routes as used hereinbefore or about from 1 to 99% of the heretofore-employed dosages. If such second medicaments are used at all, optionally, they are used in lower amounts than if the first medicament were not present, especially in subsequent dosings beyond the initial dosing with the first medicament, so as to eliminate or reduce side effects caused thereby. For instance, therapy with a CD20 antibody herein permits tapering or discontinued administration of steroid.

Combined administration herein includes co-administration, using separate formulations or a single pharmaceutical formulation, and consecutive administration in either order, wherein preferably there is a time period while both (or all) active agents simultaneously exert their biological activities.

For the retreatment method herein, where a second medicament is administered in an effective amount with an antibody set of doses, it may be administered with any set of doses, for example, only with one set of doses, or with more than one set of doses. In one embodiment, the second medicament is administered with the initial set of doses. In another embodiment, the second medicament is administered with the initial and second set of doses. In a still further embodiment, the second medicament is administered with all sets of doses.

The combined administration of a second medicament includes co-administration (concurrent administration), using separate formulations or a single pharmaceutical formulation, and consecutive administration in either order, wherein preferably there is a time period while both (or all) active agents (medicaments) simultaneously exert their biological activities.

The antibody or antagonist herein is administered by any suitable means, including parenteral, topical, subcutaneous, intraperitoneal, intrapulmonary, intranasal, and/or intralesional administration. Parenteral infusions include intramuscular, intravenous (i.v.), intraarterial, intraperitoneal, or subcutaneous administration. Intrathecal administration is also contemplated (see, e.g., US 2002/0009444, Grillo-Lopez, A concerning intrathecal delivery of a CD20 antibody). In addition, the antibody or antagonist may suitably be administered by pulse infusion, e.g., with declining doses of the antibody or antagonist. Preferably, the dosing is given intravenously or subcutaneously, and more preferably by intravenous infusion(s).

If multiple sets of doses of antibody are provided, each set of doses may be provided using the same or a different administration means. In one embodiment, each set of doses is by intravenous administration. In another embodiment, each set of doses is given by subcutaneous administration. In yet another embodiment, the sets of doses are given by both intravenous and subcutaneous administration, and the antibodies may be the same or different.

A discussion of methods of producing, modifying, and formulating such antagonists and antibodies follows.

III. Production of Antagonists and Antibodies

The methods and articles of manufacture of the present invention use, or incorporate, an antagonist or antibody that binds to a B-cell surface marker. Accordingly, methods for generating such antagonists or antibodies will be described here.

The B-cell surface marker to be used for production of, or screening for, antagonists or antibodies may be, e.g., a soluble form of the antigen or a portion thereof, containing the desired epitope. Alternatively, or additionally, cells expressing the B-cell surface marker at their cell surface, can be used to generate, or screen for, antagonists or antibodies. Other forms of the B-cell surface marker useful for generating antagonists or antibodies will be apparent to those skilled in the art. Preferably, the B-cell surface marker is the CD20 antigen.

While the preferred antagonist is an antibody, antagonists other than antibodies are contemplated herein. For example, the antagonist may comprise a small molecule antagonist optionally fused to, or conjugated with, a cytotoxic agent (such as those described herein). Libraries of small molecules may be screened against the B cell surface marker of interest herein in order to identify a small molecule that binds to that antigen. The small molecule may further be screened for its antagonistic properties and/or conjugated with a cytotoxic agent.

The antagonist may also be a peptide generated by rational design or by phage display (see, e.g., WO98/35036 published 13 Aug. 1998). In one embodiment, the molecule of choice may be a “CDR mimic” or antibody analogue designed based on the CDRs of an antibody. While such peptides may be antagonistic by themselves, the peptide may optionally be fused to a cytotoxic agent so as to add or enhance antagonistic properties of the peptide.

A description follows as to exemplary techniques for the production of antibodies used in accordance with the present invention.

(i) Polyclonal Antibodies

Polyclonal antibodies are preferably raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

(ii) Monoclonal Antibodies

Monoclonal antibodies are obtained from a population of substantially homogeneous antibodies, i.e., the individual antibodies comprising the population are identical except for possible naturally occurring mutations that may be present in minor amounts. Thus, the modifier “monoclonal” indicates the character of the antibody as not being a mixture of discrete antibodies.

For example, the monoclonal antibodies may be made using the hybridoma method first described by Kohler et al. Nature, 256:495 (1975), or may be made by recombinant DNA methods (U.S. Pat. No. 4,816,567).

In the hybridoma method, a mouse or other appropriate host animal, such as a hamster, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp.59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-1 1 mouse tumors available from the Salk Institute Cell Distribution Center, San Diego, Calif. USA, and SP-2 or X63-Ag8-653 cells available from the American Type Culture Collection, Manassas, Va. USA. Human myeloma and mouse-human heteromyeloma cell lines also have been described for the production of human monoclonal antibodies (Kozbor, J. Immunol., 133:3001 (1984); Brodeur et al. Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

The binding affinity of the monoclonal antibody can, for example, be determined by the Scatchard analysis of Munson et al. Anal. Biochem., 107:220 (1980).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

The monoclonal antibodies may also be produced recombinantly. DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce immunoglobulin protein, to obtain the synthesis of monoclonal antibodies in the recombinant host cells. Review articles on recombinant expression in bacteria of DNA encoding the antibody include Skerra et al. Curr. Opinion in Immunol., 5:256-262 (1993) and Plückthun, Immunol. Revs., 130:151-188 (1992).

In a further embodiment, antibodies or antibody fragments can be isolated from antibody phage libraries generated using the techniques described in McCafferty et al. Nature, 348:552-554 (1990). Clackson et al. Nature, 352:624-628 (1991) and Marks et al. J. Mol. Biol., 222:581-597 (1991) describe the isolation of murine and human antibodies, respectively, using phage libraries. Subsequent publications describe the production of high affinity (nM range) human antibodies by chain shuffling (Marks et al. Bio/Technology, 10:779-783 (1992)), as well as combinatorial infection and in vivo recombination as a strategy for constructing very large phage libraries (Waterhouse et al. Nuc. Acids. Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light-chain constant domains in place of the homologous murine sequences (U.S. Pat. No. 4,816,567; Morrison, et al. Proc. Natl Acad. Sci. USA, 81:6851 (1984)), or by covalently joining to the immunoglobulin coding sequence all or part of the coding sequence for a non-immunoglobulin polypeptide.

Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

(iii) Humanized Antibodies

Methods for humanizing non-human antibodies have been described in the art. Preferably, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al. Nature, 321:522-525 (1986); Riechmann et al. Nature, 332:323-327 (1988); Verhoeyen et al. Science, 239:1534-1536 (1988)), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework region (FR) for the humanized antibody (Sims et al. J. Immunol., 151:2296 (1993); Chothia et al. J. Mol. Biol., 196:901 (1987)). Another method uses a particular framework region derived from the consensus sequence of all human antibodies of a particular subgroup of light or heavy chains. The same framework may be used for several different humanized antibodies (Carter et al. Proc. Natl. Acad. Sci. USA, 89:4285 (1992); Presta et al. J. Immunol., 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, ie., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

(iv) Human Antibodies

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al. Proc. Natl. Acad. Sci. USA, 90:2551 (1993); Jakobovits et al. Nature, 362:255-258 (1993); Bruggermann et al. Year in Immuno., 7:33 (1993); and U.S. Pat. Nos. 5,591,669, 5,589,369 and 5,545,807. Alternatively, phage display technology (McCafferty et al. Nature 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage, mimics some of the properties of the B cell. Phage display can be performed in a variety of formats; for their review see, e.g., Johnson, Kevin S. and Chiswell, David J., Current Opinion in Structural Biology 3:564-571 (1993). Several sources of V-gene segments can be used for phage display. Clackson et al. Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al. J. Mol. Biol. 222:581-597 (1991), or Griffith et al. EMBO J. 12:725-734 (1993). See, also, U.S. Pat. Nos. 5,565,332 and 5,573,905.

Human antibodies may also be generated by in vitro activated B cells (see U.S. Pat. Nos. 5,567,610 and 5,229,275).

(v) Antibody Fragments

Various techniques have been developed for the production of antibody fragments. Traditionally, these fragments were derived via proteolytic digestion of intact antibodies (see, e.g., Morimoto et al. Journal of Biochemical and Biophysical Methods 24:107-117 (1992) and Brennan et al. Science, 229:81 (1985)). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al. Bio/Technology 10:163-167 (1992)). According to another approach, F(ab′)₂ fragments can be isolated directly from recombinant host cell culture. Other techniques for the production of antibody fragments will be apparent to the skilled practitioner. In other embodiments, the antibody of choice is a single chain Fv fragment (scFv). See WO 93/16185; U.S. Pat. No. 5,571,894; and U.S. Pat. No. 5,587,458. The antibody fragment may also be a “linear antibody,” e.g., as described in U.S. Pat. No. 5,641,870 for example. Such linear antibody fragments may be monospecific or bispecific.

(vi) Bispecific Antibodies

Bispecific antibodies are antibodies that have binding specificities for at least two different epitopes. Exemplary bispecific antibodies may bind to two different epitopes of the B cell surface marker. Other such antibodies may bind a first B cell marker and further bind a second B cell surface marker. Alternatively, an anti-B cell marker binding arm may be combined with an arm which binds to a triggering molecule on a leukocyte such as a T-cell receptor molecule (e.g. CD2 or CD3), or Fc receptors for IgG (FcγR), such as FcγRI (CD64), FcγRII (CD32) and FcγRIII (CD16) so as to focus cellular defense mechanisms to the B cell. Bispecific antibodies may also be used to localize cytotoxic agents to the B cell. These antibodies possess a B cell marker-binding arm and an arm that binds the cytotoxic agent (e.g. saporin, anti-interferon-α, vinca alkaloid, ricin A chain, methotrexate or radioactive isotope hapten). Bispecific antibodies can be prepared as full length antibodies or antibody fragments (e.g. F(ab′)₂ bispecific antibodies).

Methods for making bispecific antibodies are known in the art. Traditional production of full-length bispecific antibodies is based on the co-expression of two immunoglobulin heavy-chain-light-chain pairs, where the two chains have different specificities (Millstein et al. Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al. EMBO J., 10:3655-3659 (1991).

According to a different approach, antibody variable domains with the desired binding specificities (antibody-antigen combining sites) are fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light- chain binding, present in at least one of the fusions. DNAs encoding the immunoglobulin heavy-chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy-chain-light-chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al. Methods in Enzymology, 121:210 (1986).

According to another approach described in U.S. Pat. No. 5,731,168, the interface between a pair of antibody molecules can be engineered to maximize the percentage of heterodimers that are recovered from recombinant cell culture. The preferred interface comprises at least a part of the CH3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al. Science, 229: 81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Recent progress has facilitated the direct recovery of Fab′-SH fragments from E. coli, which can be chemically coupled to form bispecific antibodies. Shalaby et al. J. Exp. Med., 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al. J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al. Proc. Natl. Acad. Sci. USA, 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al. J. Immunol., 152:5368 (1994).

Antibodies with more than two valencies are contemplated. For example, trispecific antibodies can be prepared. Tutt et al. J. Immunol. 147: 60 (1991).

IV. Conjugates and Other Modifications of the Antagonist or Antibody

The antagonist or antibody used in the methods or included in the articles of manufacture herein is optionally conjugated to another agent, such as a cytotoxic agent, or cytokine (for example IL2; see for example, WO2005/016969).

Conjugation will ordinarily be achieved through a covalent linkage, the precise nature of which will be determined by the targeting molecule and the linking site on the CD20 antagonist or antibody polypeptide. Typically, a non-peptidic agent is modified by the addition of a linker that allows conjugation to CD20 antagonist or antibody through its amino acid side chains, carbohydrate chains, or reactive groups introduced on CD20 antagonist or antibody by chemical modification. For example, a drug may be attached through the ε-amino group of a lysine residue, through a free α-amino group, by disulfide exchange to a cysteine residue, or by oxidation of the 1,2- diols in a carbohydrate chain with periodic acid to allow attachment of drugs containing various nucleophiles through a Schiff-base linkage. See, for example, U.S. Pat. No. 4,256,833. Protein modifying agents include amine-reactive reagents (e.g., reactive esters, isothiocyantates, aldehydes, and sulfonyl halides), thiol-reactive reagents (e.g., haloacetyl derivatives and maleimides), and carboxylic acid- and aldehyde-reactive reagents. CD20 antagonist or antibody polypeptides can be covalently joined to peptidic agents through the use of bifunctional cross-linking reagents. Heterobifunctional reagents are more commonly used and permit the controlled coupling of two different proteins through the use of two different reactive moieties (e.g., amine-reactive plus thiol, iodoacetamide, or maleimide). The use of such linking agents is well known in the art. See, for example, Brinkley, supra, and U.S. Pat. No. 4,671,958. Peptidic linkers can also be employed. In the alternative, a CD20 antagonist or antibody polypeptide can be linked to a peptidic moiety through preparation of a fusion polypeptide.

Examples of further bifunctional protein coupling agents include N-succinimidyl-3-(2-pyridyldithiol) propionate (SPDP), succinimidyl-4-(N-maleimidomethyl) cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene).

Alternatively, a fusion protein comprising the antagonist or antibody and agent may be made, e.g. by recombinant techniques or peptide synthesis.

Other modifications of the antagonist or antibody are contemplated herein. For example, the antagonist or antibody may be linked to one of a variety of nonproteinaceous polymers, e.g., polyethylene glycol, polypropylene glycol, polyoxyalkylenes, or copolymers of polyethylene glycol and polypropylene glycol.

The antagonist or antibody disclosed herein may also be formulated as liposomes. Liposomes containing the antagonist or antibody are prepared by methods known in the art, such as described in Epstein et al. Proc. Natl. Acad. Sci. USA, 82:3688 (1985); Hwang et al. Proc. Natl. Acad. Sci. USA, 77:4030 (1980); U.S. Pat. Nos. 4,485,045 and 4,544,545; and WO97/38731 published Oct. 23, 1997. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of an antibody of the present invention can be conjugated to the liposomes as described in Martin et aL J. BioL Chem. 257: 286-288 (1982) via a disulfide interchange reaction. A chemotherapeutic agent is optionally contained within the liposome. See Gabizon et al. J. National Cancer Inst. 81(19): 1484 (1989).

Amino acid sequence modification(s) of protein or peptide antagonist or antibodies described herein are contemplated. For example, it may be desirable to improve the binding affinity and/or other biological properties of the antagonist or antibody. Amino acid sequence variants of the antagonist or antibody are prepared by introducing appropriate nucleotide changes into the antagonist or antibody nucleic acid, or by peptide synthesis. Such modifications include, for example, deletions from, and/or insertions into and/or substitutions of, residues within the amino acid sequences of the antagonist or antibody. Any combination of deletion, insertion, and substitution is made to arrive at the final construct, provided that the final construct possesses the desired characteristics. The amino acid changes also may alter post-translational processes of the antagonist or antibody, such as changing the number or position of glycosylation sites.

A useful method for identification of certain residues or regions of the antagonist or antibody that are preferred locations for mutagenesis is called “alanine-scanning mutagenesis” as described by Cunningham and Wells Science, 244:1081-1085 (1989). Here, a residue or group of target residues are identified (e.g., charged residues such as arg, asp, his, lys, and glu) and replaced by a neutral or negatively charged amino acid (most preferably alanine or polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed antagonist or antibody variants are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antagonist or antibody with an N-terminal methionyl residue or the antagonist or antibody fused to a cytotoxic polypeptide. Other insertional variants of the antagonist or antibody molecule include the fusion to the N- or C-terminus of the antagonist or antibody of an enzyme, or a polypeptide that increases the serum half-life of the antagonist or antibody.

Another type of variant is an amino acid substitution variant. These variants have at least one amino acid residue in the antagonist or antibody molecule replaced by different residue. The sites of greatest interest for substitutional mutagenesis of antibody antagonists include the hypervariable regions, but FR alterations are also contemplated. Conservative substitutions are shown in Table 4 under the heading of “preferred substitutions”. If such substitutions result in a change in biological activity, then more substantial changes, denominated “exemplary substitutions” in Table 4, or as further described below in reference to amino acid classes, may be introduced and the products screened.

TABLE 4

| Original | Exemplary | Preferred |
|---|---|---|
| Residue | Substitutions | Substitutions |
| Ala (A) | val; leu; ile | val |
| Arg (R) | lys; gln; asn | lys |
| Asn (N) | gln; his; asp, lys; arg | gln |
| Asp (D) | glu; asn | glu |
| Cys (C) | ser; ala | ser |
| Gln (Q) | asn; glu | asn |
| Glu (E) | asp; gln | asp |
| Gly (G) | Ala | ala |
| His (H) | asn; gln; lys; arg | arg |
| Ile (I) | leu; val; met; ala; | leu |
| | phe; Norleucine | |
| Leu (L) | norleucine; ile; val; | ile |
| | met; ala; phe | |
| Lys (K) | arg; gln; asn | arg |
| Met (M) | leu; phe; ile | leu |
| Phe (F) | leu; val; ile; ala; tyr | tyr |
| Pro (P) | Ala | ala |
| Ser (S) | Thr | thr |
| Thr (T) | Ser | ser |
| Trp (W) | tyr; phe | tyr |
| Tyr (Y) | trp; phe; thr; ser | phe |
| Val (V) | ile; leu; met; phe; | leu |
| | ala; Norleucine | |

Substantial modifications in the biological properties of the antagonist or antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;

(2) neutral hydrophilic: cys, ser, thr;

(3) acidic: asp, glu;

(4) basic: asn, gln, his, lys, arg;

(5) residues that influence chain orientation: gly, pro; and

(6) aromatic: trp, tyr, phe.

Non-conservative substitutions will entail exchanging a member of one of these classes for another class.

Any cysteine residue not involved in maintaining the proper conformation of the antagonist or antibody also may be substituted, generally with serine, to improve the oxidative stability of the molecule and prevent aberrant crosslinking. Conversely, cysteine bond(s) may be added to the antagonist or antibody to improve its stability (particularly where the antagonist or antibody is an antibody fragment such as an Fv fragment).

A particularly preferred type of substitutional variant involves substituting one or more hypervariable region residues of a parent antibody. Generally, the resulting variant(s) selected for further development will have improved biological properties relative to the parent antibody from which they are generated. A convenient way for generating such substitutional variants is affinity maturation using phage display. Briefly, several hypervariable region sites (e.g. 6-7 sites) are mutated to generate all possible amino substitutions at each site. The antibody variants thus generated are displayed in a monovalent fashion from filamentous phage particles as fusions to the gene In product of M13 packaged within each particle. The phage-displayed variants are then screened for their biological activity (e.g. binding affinity) as herein disclosed. In order to identify candidate hypervariable region sites for modification, alanine-scanning mutagenesis can be performed to identify hypervariable region residues contributing significantly to antigen binding. Alternatively, or in additionally, it may be beneficial to analyze a crystal structure of the antigen-antibody complex to identify contact points between the antibody and antigen. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the antagonist or antibody alters the original glycosylation pattern of the antagonist or antibody. By altering is meant deleting one or more carbohydrate moieties found in the antagonist or antibody, and/or adding one or more glycosylation sites that are not present in the antagonist or antibody.

Glycosylation of polypeptides is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antagonist or antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antagonist or antibody (for O-linked glycosylation sites).

Where the antagonist or antibody comprises an Fc region, the carbohydrate attached thereto may be altered. For example, antibodies with a mature carbohydrate structure that lacks fucose attached to an Fc region of the antibody are described in US Pat Appl No US 2003/0157108 A1, Presta, L. See also US 2004/0093621 A1 (Kyowa Hakko Kogyo Co., Ltd). Antibodies with a bisecting N-acetylglucosamine (GlcNAc) in the carbohydrate attached to an Fc region of the antibody are referenced in WO03/011878, Jean-Mairet et al. and U.S. Pat. No. 6,602,684, Umana et al. Antibodies with at least one galactose residue in the oligosaccharide attached to an Fc region of the antibody are reported in WO97/30087, Patel et al. See, also, W)98/58964 (Raju, S.) and WO99/22764 (Raju, S.) concerning antibodies with altered carbohydrate attached to the Fc region thereof.

The preferred glycosylation variant herein comprises an Fc region, wherein a carbohydrate structure attached to the Fc region lacks fucose. Such variants have improved ADCC function. Optionally, the Fc region further comprises one or more amino acid substitutions therein which further improve ADCC, for example, substitutions at positions 298, 333, and/or 334 of the Fc region (Eu numbering of residues). Examples of publications related to “defucosylated” or “fucose-deficient” antibodies include: US Pat. Appl. No. US 2003/0157108 Al, Presta, L; WO 00/61739A1; WO01/29246A1; US2003/0115614A1; US2002/0164328A1; US2004/0093621A1; US2004/0132140A1; US2004/0110704A1; US2004/0110282A1; US2004/0109865A1; W003/085119A1; W003/084570A1; Okazaki et al. J. Mol. Biol. 336:1239-1249 (2004); Yamane-Ohnuki et al. Biotech. Bioeng. 87: 614 (2004). Examples of cell lines producing defucosylated antibodies include Lec13 CHO cells deficient in protein fucosylation (Ripka et al. Arch. Biochem. Biophys. 249:533-545 (1986); US Pat Appl No US 2003/0157108 Al, Presta, L; and WO 2004/056312 A1, Adams et al., especially at Example 11), and knockout cell lines, such as alpha-1,6-fucosyltransferase gene, FUT8, knockout CHO cells (Yamane-Ohnuki et al. Biotech. Bioeng. 87: 614 (2004)).

Nucleic acid molecules encoding amino acid sequence variants of the antagonist or antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antagonist or antibody.

It may be desirable to modify the antagonist or antibody of the invention with respect to effector function, e.g. so as to enhance antigen-dependent cell-mediated cyotoxicity (ADCC) and/or complement dependent cytotoxicity (CDC) of the antagonist or antibody. This may be achieved by introducing one or more amino acid substitutions in an Fc region of an antibody antagonist or antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al. J. Exp Med. 176:1191-1195 (1992) and Shopes, B. J. Immunol. 148:2918-2922 (1992). Homodimeric antibodies with enhanced anti-tumor activity may also be prepared using heterobifunctional cross-linkers as described in Wolff et al. Cancer Research 53:2560-2565 (1993). Alternatively, an antibody can be engineered which has dual Fc regions and may thereby have enhanced complement lysis and ADCC capabilities. See Stevenson et al. Anti-Cancer Drug Design 3:219-230 (1989).

WO00/42072 (Presta, L.) describes antibodies with improved ADCC function in the presence of human effector cells, where the antibodies comprise amino acid substitutions in the Fc region thereof. Preferably, the antibody with improved ADCC comprises substitutions at positions 298, 333, and/or 334 of the Fc region (Eu numbering of residues). Preferably the altered Fc region is a human IgGI Fc region comprising or consisting of substitutions at one, two or three of these positions. Such substitutions are optionally combined with substitution(s) which increase C1q binding and/or CDC.

Antibodies with altered C1q binding and/or complement dependent cytotoxicity (CDC) are described in WO99/51642, U.S. Pat. No. 6,194,551B1, U.S. Pat. No. 6,242,195B1, U.S. Pat. No. 6,528,624B1 and U.S. Pat. No. 6,538,124 (Idusogie et al.). The antibodies comprise an amino acid substitution at one or more of amino acid positions 270, 322, 326, 327, 329, 313, 333 and/or 334 of the Fc region thereof (Eu numbering of residues). Substitution of one or more residues at positions 326, 327, 333 and/or 334 can improve C1q binding and/or CDC function.

To increase the serum half life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

Antibodies with improved binding to the neonatal Fc receptor (FcRn), and increased half-lives, are described in WO00/42072 (Presta, L.) and US2005/0014934A1 (Hinton et al.). These antibodies comprise an Fc region with one or more substitutions therein which improve binding of the Fc region to FcRn. For example, the Fc region may have substitutions at one or more of positions 238, 250, 256, 265, 272, 286, 303, 305, 307, 311, 312, 314, 317, 340, 356, 360, 362, 376, 378, 380, 382, 413, 424, 428 or 434 (Eu numbering of residues). The preferred Fc region-comprising antibody variant with improved FcRn binding comprises amino acid substitutions at one, two or three of positions 307, 380 and 434 of the Fc region thereof (Eu numbering of residues).

Engineered antibodies with three or more (preferably four) functional antigen binding sites are also contemplated (US Appln No. US2002/0004587 Al, Miller et aL).

V. Pharmaceutical Formulations

Therapeutic formulations of the antagonist or antibody used in accordance with the present invention are prepared for storage by mixing an antagonist or antibody having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Exemplary anti-CD20 antibody formulations are described in WO 1998/56418. This publication describes a liquid multidose formulation comprising 40 mg/mL rituximab, 25 mM acetate, 150 mM trehalose, 0.9% benzyl alcohol, 0.02% polysorbate 20 at pH 5.0 that has a minimum shelf life of two years storage at 2-8° C. Another anti-CD20 formulation of interest comprises 10 mg/mL rituximab in 9.0 mg/mL sodium chloride, 7.35 mg/mL sodium citrate dihydrate, 0.7 mg/mL polysorbate 80, and Sterile Water for Injection, pH 6.5.

Lyophilized formulations adapted for subcutaneous administration are described in U.S. Pat. No. 6,267,958 (Andya et al.). Such lyophilized formulations may be reconstituted with a suitable diluent to a high protein concentration and the reconstituted formulation may be administered subcutaneously to the subject to be treated herein.

The formulation herein may also contain more than one active compound (a second medicament as noted above) as necessary, preferably those with complementary activities that do not adversely affect each other. The type and effective amounts of such medicaments depend, for example, on the amount of antagonist or antibody present in the formulation, and clinical parameters of the subjects being treated. The preferred such medicaments are noted above.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antagonist or antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes.

VI. Articles of Manufacture

In another embodiment of the invention, articles of manufacture containing materials useful for the treatment of a IBD described above are provided. In one aspect, the article of manufacture comprises (a) a container comprising an antagonist (e.g. an antibody) that binds to a B-cell surface marker (e.g., CD20), optionally in a pharmaceutically acceptable carrier or diluent; and (b) a package insert with instructions for treating a IBD in a human subject.

In all of these aspects, the package insert is on or associated with the container. Suitable containers include, for example, bottles, vials, syringes, etc. The containers may be formed from a variety of materials such as glass or plastic. The container holds or contains a composition that is effective for treating the IBD and may have a sterile access port (for example the container may be an intravenous solution bag or a vial having a stopper pierceable by a hypodermic injection needle). At least one active agent in the composition is the antagonist or antibody. The label or package insert indicates that the composition is used for treating a human subject eligible for treatment, e.g., one having or predisposed to IBD, including moderate-severe IBD or UC, with specific guidance regarding dosing amounts and intervals of antagonist or antibody and any other medicament being provided. The article of manufacture may further comprise an additional container comprising a pharmaceutically acceptable diluent buffer, such as bacteriostatic water for injection (BWFI), phosphate-buffered saline, Ringer's solution, and/or dextrose solution. The article of manufacture may further include other materials desirable from a commercial and user standpoint, including other buffers, diluents, filters, needles, and syringes.

The article of manufacture herein optionally further comprises a container comprising a second medicament, wherein the antibody is a first medicament, and which article further comprises instructions on the package insert for treating the subject with the second medicament, in an effective amount. The second medicament may be any of those set forth above, with an exemplary second medicament being an aminosalicylate, an oral corticosteroid, 6-mercaptopurine (6-MP), and azathioprine.

Further details of the invention are illustrated by the following non-limiting Example. The disclosures of all citations in the specification are expressly incorporated herein by reference.

### EXAMPLE 1

### Therapy of IBD

This is an evaluation of rituximab in human subjects with active UC as defined in the inclusion criteria. Gene microarray data have demonstrated that B-cell genes and CD20 expression are upregulated in human UC. This example provides a protocol for therapy of UC subjects.

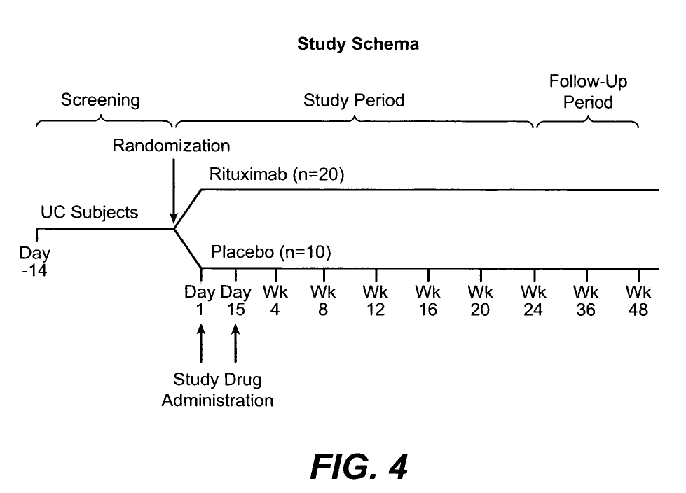

Study schemia for this protocol depicted in FIG. 4.

Therapy herein includes a screening period of about 2 weeks, a study period of about 24 weeks, and a follow-up period of 24 weeks. Rigorous evaluations of safety will be performed throughout the study. The screening period from Day -14 to Day 0 includes a medical history, physical examination, laboratory assessments, collection of diary data, and a flexible sigmoidoscopy with biopsies to confirm active disease and determine the baseline Disease Activity Index (DAI) score. The DAI score is used to identify potential subjects for enrollment in the study and to assess the clinical activity of rituximab.

Subjects will receive a 1 gram intravenous (IV) infusion of rituximab (or placebo) on Days 1 and 15. All subjects will continue on stable doses of one or more of the following through at least Week 8: an aminosalicylate, an oral corticosteroid, 6-MP, and/or azathioprine. Safety monitoring with periodic laboratory and physical examination evaluations will be performed at all study visits. After the Day 1 and 15 visits, subjects will have scheduled visits every 4 weeks to Week 24 and then every 3 months to Week 48. In addition, subjects will return on Day 2 and Day 16 for pharmacokinetic (PK) samples only. Repeat flexible sigmoidoscopy with biopsies will be performed at Week 8 for histology, disease assessment, and evaluation of mucosal B-cell depletion. An additional sigmoidoscopy with biopsies will be performed at Week 24 to evaluate disease status and recovery of B-cell depletion in colonic mucosa. The histologic assessment of inflammation in the biopsy specimens will be scored according to a standardized scale (Geboes et al., Gut 47:404409 (2000)).

A DAI score will be calculated at Week 8 to determine the proportion of subjects achieving disease remission. The DAI score will also be calculated at the Week 24 visit. A clinical assessment will be performed at visits when a flexible sigmoidoscopy is not performed (i.e., Days 1 and 15 and Weeks 4, 12, 16, 20, 36, and 48). Subjects will be followed by the investigator and the Sponsor until Week 48 or until B-cell recovery, whichever is longer. B-cell recovery is defined as B-cell levels that have returned to baseline (Day 1) or to the lower limit of normal.

This example provides an evaluation of the safety and tolerability of rituximab in adult subjects with active UC. The primary safety outcome measure is the frequency of targeted events of protocol-defined UC exacerbations (worsening of disease).

B-cell evaluation will continue until Week 48 or B-cell recovery, whichever is longer.

This example also evaluates the therapeutic clinical activity of rituximab in UC using the DAI scoring system as defined below. A DAI scoring system for assessment of UC activity has been used in pivotal clinical trials (Schroeder et al, N Engl J Med 317:1625-1629 (1987)). Remission of signs and symptoms of active disease, as evidenced by cessation of rectal bleeding and healing of friable mucosa, has been chosen as a secondary endpoint for clinical activity. Duration of remission will also be measured. The flexible sigmoidoscopy results and DAI score at Week 24 and clinical follow-up to Week 48 will enable evaluation of the duration of the therapeutic effect.

Outcome Measures

Primary Safety Outcome Measure

The primary safety outcome measure is the targeted adverse event frequency of protocol-defined UC exacerbations occurring during the study period (Day 1 to Week 24).

A protocol-defined UC exacerbation must satisfy one or more of the following criteria:
A ≧3-point increase in DAI score
Suspected or impending toxic megacolon
Need for hospitalization for an exacerbation of UC
In the clinical judgment of the investigator, medically significant worsening of disease
   Secondary Outc6me Measures
   Secondary outcome measures are the following:
Other safety outcome measures
Incidence of serious infections, defined as infections requiring hospitalization or IV antibiotics
Incidence of all adverse events (serious and non-serious) graded according to National Cancer Institute Common Toxicity Criteria of Adverse Events (NCI-CTCAE), Version 3.0
Incidence of clinical laboratory abnormalities
Proportion of subjects who achieve disease remission at Week 8
Disease remission is defined as a sigmoidoscopy score of 0 or 1 (no friability) and rectal bleeding score of 0.
Proportion of subjects achieving a clinical response at Week 8
Clinical response is defined as a reduction of >3 points in the DAI score.
Time to disease remission
Duration of disease remission as determined by the investigator
Change from baseline during the study period in results of the Inflammatory Bowel Disease Questionnaire (IBDQ)

The effects of rituximab on several pharmacodynamic markers will be examined by comparing blood, serum, and tissue samples at baseline and during treatment. These assessments will be as follows:
Blood lymphocyte panel with B-cell count (CD19+ and other B-cell phenotype subsets)
Serum Ig levels (total, IgA, IgG, and IgM)
Antibodies specific to UC (p-ANCA)
B-cell depletion in colonic biopsies, as measured by immunohistochemistry (IHC)
   Subjects
   Inclusion Criteria
   Subjects must meet the following criteria to be eligible for study entry:
Written informed consent
Age 18-75 years and capable of understanding study procedures
Diagnosis of UC >6 months at screening
≧20 cm of active disease at screening sigmoidoscopy
Active disease, as defined by a DAI score between ≧6 and ≦11, with ≧2 for rectal bleeding and ≧2 for flexible sigmoidoscopy at screening
Treatment with oral corticosteroids for UC within 2 years prior to screening
Treatment intensity should have been equal to or greater than a prednisone equivalent dose of 20 mg/day for at least 2 weeks' duration.
Colonoscopy within the past 2 years for extent of disease and to exclude polyps
Colonoscopy with appropriate biopsies to exclude dysplasia within 1 year prior to screening if UC disease>10 years
For subjects of reproductive potential (males and females), use a reliable means of contraception (e.g., hormonal contraceptive, patch, vaginal ring, intrauterine device, physical barrier) during study treatment and for 1 year following the last dose of study drug
Withdrawal of all previous investigative biologic therapy (e.g., etanercept, infliximab, adalimumab, rituximab) at least 15 weeks prior to randomization
Current treatment with one or more of the following therapies on a stable dose for the indicated period prior to baseline (Day 1):
   Aminosalicylate, stable dose for ≧3 weeks
   Oral corticosteroids, stable dose for >2 weeks
   6-MP treatment for a 3-month period, with a stable dose for ≧4 weeks
   Azathioprine treatment for a 3-month period, with a stable dose for ≧4 weeks
For the therapies listed above that have been used previously but not currently at Day 1, subjects need to have discontinued aminosalicylates for ≧2 weeks and to have discontinued azathioprine treatment, 6-MP treatment, or oral corticosteroids for ≧4 weeks prior to baseline.
   Exclusion Criteria
   Subjects who meet any of the following criteria will be excluded from study entry:
Severe colitis as evidenced by investigator judgment that the subject is likely to require a colectomy or institution of a calcineurin inhibitor within 12 weeks of baseline (Day 1)
Clinical suspicion or radiographic evidence of colonic perforation or toxic Megacolon
History of primary sclerosing cholangitis
History of colonic dysplasia and/or adenomatous polyps in the colon
Treatment with cyclosporine, tacrolimus, sirolimus, methotrexate, or mycophenolate mofetil within 8 weeks prior to screening
Treatment with a topical rectal preparation within 2 weeks prior to screening
Use of nonsteroidal anti-inflammatory drugs (NSAIDs) other than low-dose aspirin within 4 weeks prior to baseline
Positive stool for ova or parasites, positive stool culture for pathogens, or positive stool toxin assay for Clostridium difficile at screening
Receipt/treatment with any live vaccines within 4 weeks prior to randomization
Previous treatment with any non-biologic cell-depleting therapies such as ADACOLUMN®
History of colonic or small bowel obstruction or resection
Use of antidiarrheal agents during the screening period
History of hepatitis B or C
   Exclusions for General Safety
History of severe allergic or anaphylactic reactions to humanized, chimeric, or fully human antibodies or murine monoclonal antibodies
Significant cardiac or pulmonary disease (including obstructive pulmonary disease)
Evidence of significant uncontrolled concomitant diseases such as cardiovascular disease or nervous system, pulmonary, renal, hepatic, endocrine, or gastrointestinal disorders
Known active bacterial, viral, fungal, mycobacterial, or other infection (including tuberculosis or atypical mycobacterial disease, but excluding fungal infections of nail beds) or any major episode of infection requiring hospitalization or treatment with IV antibiotics within 4 weeks of screening or oral antibiotics within 2 weeks of screening
History of recurrent significant infection or recurrent bacterial infections
Primary or secondary immunodeficiency (history of or currently active), including HIV
History of cancer, including solid tumors and hematologic malignancies (except basal cell and squamous cell carcinomas of the skin that have been excised and cured)
Pregnant women or nursing (breast feeding) mothers
History of alcohol, drug, or chemical abuse within the 6 months prior to screening
Lack of peripheral venous access
   Laboratory Exclusion Criteria (at screening)
Serum creatinine >1.4 mg/dL for women or ≧1.6 mg/dL for men
Aspartate aminotransferase (AST) or alanine aminotransferase (ALT) >2.5 times upper limit of normal
Platelet count <100,000/μL
Hemoglobin <8.5 g/dL
Neutrophils <1 500/μL
Lymphocyte count <100/μL
Positive hepatitis B or C serology
IgG <5.65 mg/μL
IgM <0.55 mg/mL
B-cell count <1.1%
Electrocardiogram (ECG) showing a significant cardiac abnormality that the Principal Investigator determines may jeopardize the subject's health by participating in this study
   Study Treatment
   Formulation

Rituximab is formulated for IV administration as a sterile product in 9.0 mg/mL sodium chloride, 0.7 mg/mL polysorbate 80, 7.35 mg/mL sodium citrate dehydrate, and Sterile Water for Injection (pH 6.5). The antibody is supplied for market use in 10-mL and 50-mL vials at a concentration of 10.0 mg/mL. The 10-mL vials contain 100 mg of antibody. The 50-mL vials contain 500 mg of antibody. No preservative is used because the vial is designed for single use. Study sites will be supplied 50-mL vials of 500 mg of rituximab and 50-mL vials of matching placebo.

Dosage, Administration, and Storage

Study treatment will consist of 1 gram of rituximab or placebo equivalent administered IV on Days 1 and 15. Subjects will receive prophylactic treatment with acetaminophen (1 g) and diphenhydramine HCI (50 mg), or their equivalent, by mouth 30-60 minutes prior to the start of each infusion. Subjects may be hospitalized for observation, particularly for their first infusion, at the discretion of the investigator. Rituximab must be administered under close supervision, and full resuscitation facilities must be immediately available. If a protocol-defined UC exacerbation occurs prior to the second infusion, the second infusion will be held.

Rituximab solutions for infusion are stable at 2° C.-8° C. (36° F.-46° F.) for 24 hours and at room temperature for an additional 24 hours. Do not use beyond the expiration date stamped on the carton. No incompatibilities between rituximab and polyvinyl chloride or polyethylene bags have been observed.

Concomitant and Excluded Therapies

Prior to baseline (Day 1), all subjects will be on stable doses of an aminosalicylate, an oral corticosteroid, 6-MP, and/or azathioprine for variable periods prior to baseline. Throughout the study and follow-up periods, subjects should maintain their constant dose of aminosalicylate, 6-MP, and/or azathioprine. Oral corticosteroid doses should remain stable until after Week 8, if medically acceptable. Tapering should be instituted if medically indicated after Week 8.

Therapies for disease conditions other than UC may be continued, except as noted below. Use of live virus or bacteria vaccines is prohibited from Day -28 through the end of the study period. These vaccines may include, but are not limited to, measles, mumps, rubella, polio, bacille Calmette-Guerin, yellow fever, and TY21a typhoid. Vaccines that do not contain live organisms (e.g., influenza, Pneumovax®, tetanus) are not prohibited, but may not be effective. It is recommended that a subject's vaccination record and possible requirements be reviewed, and, if necessary, any required vaccination/booster be given at least 28 days prior to initiation of study drug treatment.

Treatment with cyclosporine, tacrolimus, sirolimus, methotrexate, or mycophenolate mofetil is prohibited within 8 weeks of screening and during the study. Cyclosporine in any formulation may be used at the discretion of the investigator as a rescue medication for a protocol-defined UC exacerbation. If rescue medication is needed prior to Week 8, the subject will be considered a non-responder but should continue scheduled study visits.

Other excluded medications during the study period are as follows:
Antibiotics to treat UC
Antibiotics may be used to treat infections as medically indicated but not as a therapy of UC.
NSAIDs, with the exception of low-dose aspirin for cardiovascular prophylaxis
Topical rectal therapies for UC
Antidiarrheals
Laxatives
Bile-acid binders such as cholestyramine
Investigational drugs or treatments is prohibited
   Assay Methods

Serum samples will be obtained for PK and HACA analyses at timepoints according to the assessment schedule.

The rituximab PK enzyme-linked immunosorbent assay (ELISA) will be used to measure rituximab level in human serum samples.

The rituximab HACA ELISA is a bridging assay, which uses rituximab as the capturing reagent and biotinylated-rituximab and streptavidin-HRP for detection. The assay uses a calibrator curve prepared with affinity-purified polyclonal goat antibodies to rituximab; therefore, results from this assay are reported relative to this polyclonal antibody in terms of relative units.

All p-ANCA analysis will be performed by a central laboratory. Indirect immunofluorescence will be used to determine the presence of ANCAs. In addition, ELISA assays may be used to determine the specificity of ANCA for myeloperoxidase or other relevant antigens as determined by the central laboratory.

Clinical Activitv Analyses

The clinical activity of rituximab in UC will be evaluated. The proportions of subjects experiencing disease remission and the proportions of subjects with clinical responses at Week 8 will be estimated and corresponding 95% confidence intervals will be generated for each treatment arm. Treatment differences and 95% confidence intervals will be provided.

Duration of disease remission will be summarized by treatment arm. Median time to disease remission response will be summarized for each treatment arm using the Kaplan-Meier method for descriptive purposes only.

Subjects with active UC treated with the rituximab antibody as described above, will experience an improvement in the signs and symptoms of UC, including disease remission and/or clinical response (achieved by week 8), attainment of a sigmoidoscopy score of 0 or 1, and rectal bleeding score of 0, a reduction in DAI score (by greater than or equal to 3 points), a reduction in B-cells in colonic mucosa, and/or a reduction in p-ANCA antibody level.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A method for treating moderate-severe inflammatory bowel disease (IBD) in a human subject comprising administering to the subject an effective amount of a CD20 antibody, wherein administration of the antibody results in a clinical response or disease remission.

2. The method of claim 1 wherein the IBD is ulcerative colitis (UC).

3. The method of claim 1 wherein the IBD is Crohn's disease.

4. The method of claim 1 wherein the subject has active IBD.

5. The method of claim 1 wherein administration of the antibody results in disease remission.

6. The method of claim 5 wherein remission is achieved at about week 8.

7. The method of claim 5 wherein administration of the antibody results in a sigmoidoscopy score of 0 or 1 and rectal bleeding score of 0.

8. The method of claim 1 wherein administration of the antibody results in a clinical response.

9. The method of claim 8 wherein the clinical response is achieved at about week 8.

10. The method of claim 8 wherein administration of the antibody reduces disease activity index (DAI) score.

11. The method of claim 10 wherein the DAI score, scored using the scoring system in Table 2 herein, is reduced by greater than or equal to 3 points.

12. The method of claim 1 wherein administration of the antibody reduces B cells in colonic mucosa.

13. The method of claim 1 wherein the antibody is a chimeric, human, or humanized antibody.

14. The method of claim 1 wherein the antibody comprises rituximab.

15. The method of claim 1 wherein the antibody comprises humanized 2H7.

16. The method of claim 1 wherein the antibody comprises 2F2 (huMax-CD20).

17. The method of claim 1 wherein the antibody is a naked antibody.

18. The method of claim 1 wherein the antibody is conjugated with another molecule.

19. The method of claim 1 wherein the antibody is administered as a dose in the range from about 200 mg to 2000 mg at a frequency of about one to four doses within a period of about one month.

20. The method of claim 19 wherein the dose is in the range from about 500 mg to 1500 mg.

21. The method of claim 19 wherein the dose is in the range from about 750 mg to 1200 mg.

22. The method of claim 19 wherein the antibody is administered in one or two doses.

23. The method of claim 19 wherein the antibody is administered within a period of about 2 to 3 weeks.

24. The method of claim 23 wherein the period is about two weeks.

25. The method of claim 1 wherein the antibody is administered intravenously.

26. The method of claim 1 wherein the antibody is administered subcutaneously.

27. The method of claim 1 wherein a second medicament is administered in an effective amount, wherein the CD20 antibody is a first medicament.

28. The method of claim 27 wherein the second medicament is more than one medicament.

29. The method of claim 27 wherein the second medicament is selected from the group consisting of an aminosalicylate, an oral corticosteroid, 6-mercaptopurine (6-MP) and azathioprine.

30. The method of claim 27 wherein the second medicament is administered in a lower amount than is used if the CD20 antibody is not administered to a subject treated with the second medicament.

31. The method of claim 1 wherein the subject has never been previously treated with a CD20 antibody.

32. The method of claim 1 wherein the subject is not suffering from a B cell malignancy.

33. The method of claim 1 wherein the subject is not suffering from an autoimmune disease, other than IBD.

34. A method for treating inflammatory bowel disease (IBD) in a human subject with active IBD comprising administering only one or two doses of a CD20 antibody to the subject, wherein disease remission or clinical response is achieved upon administration of the one or two doses of the CD20 antibody.

35. The method of claim 34 wherein the one or two doses are administered intravenously (IV).

36. The method of claim 34 wherein the one or two doses are administered subcutaneously (SQ).

37. The method of claim 34 wherein two intravenous doses are administered, wherein each of the two doses is in the range from about 200 mg to about 2000 mg.

38. A method for treating inflammatory bowel disease (IBD) in a human subject with active IBD comprising administering to the subject an effective amount of a CD20 antibody and further comprising administering to the subject an effective amount of a second medicament selected from the group consisting of an aminosalicylate, an oral corticosteroid, 6-mercaptopurine (6-MP) and azathioprine.

39. A method for reducing a disease activity index (DAI) score in a human subject with active ulcerative colitis (UC) comprising administering a CD20 antibody to the subject in an amount effective to reduce DAI score.

40. The method of claim 39 wherein the DAI score, scored using the scoring system in Table 2 herein, is reduced by greater than or equal to 3 points.

41. The method of claim 1 wherein the subject has an atypical level of perinuclear antineutrophil cytoplasmic antibody (p-ANCA), or anti-human tropomyosin isoform 5 (hTM5) autoantibody.

42. An article of manufacture comprising:
i. a container comprising a CD20 antibody; and
ii. a package insert with instructions for treating inflammatory bowel disease (IBD) in a human subject, wherein the instructions indicate that an effective amount of the CD20 antibody is administered to the human subject.
